(19) United States
(12) Patent Application Publication
(10) Pub. No.: US 2006/0135419 A1
(43) Pub. Date: Jun. 22, 2006

(54) Proteins involved in the regulation of energy homeostasis

(75) Inventors: f Meise, Homer (DE); Karsten Eulenberg, Bovenden (DE); Tri Nguyen, Gottingen (DE); Theodoros Tsetsenis, Monterotondo (RO)

Correspondence Address: ROTHWELL, FIGG, ERNST & MANBECK, P.C., 1425 K STREET, N.W., SUITE 800, WASHINGTON, DC, 20005, US

(73) Assignee: DeveloGen Aktiengesellschaft fuer entwick- lungsbiologische Forschung, Goettingen (DE)

(21) Appl. No.: 10/537,303

(22) PCT Filed: Dec. 3, 2003
(86) PCT No.: PCT/EP03/13655

§ 371 Date: Jun. 2, 2005

(30) Foreign Application Priority Data
Dec. 3, 2002 (EP) 02 026 921.3

(51) Int. Cl. A61K 38/17; A61K 48/00

(52) U.S. Cl. 514/12; 514/44

(57) ABSTRACT

The prevent invention discloses PRL-1 homologous proteins regulating the energy homeostasis and the metabolism of triglycerides, and polynucleotides, which identify and encode the proteins disclosed in this invention. The invention also relates to the use of these sequences in the diagnosis, study, prevention, and treatment of metabolic diseases and disorders.

This invention relates to the use of nucleic acid sequences encoding mammalian protein tyrosine phosphatase (referred to as Prl-1, Prl-2, or Prl-3; PRL-1 homologous protein), and the polypeptides encoded thereby and to the use thereof or effectors of Prl-1, Prl-2, or Prl-3 in the diagnosis, study, prevention, and treatment of diseases and disorders related to body-weight regulation, for example, but not limited to, metabolic diseases or dysfunctions such as obesity as well as related disorders such as diabetes, e.g. type II diabetes.

There are several metabolic diseases of human and animal metabolism, e.g., obesity and severe weight loss, that relate to energy imbalance where caloric intake versus energy expenditure is imbalanced. Obesity is one of the most prevalent metabolic disorders in the world. It is still a poorly understood human disease that becomes as a major health problem more and more relevant for western society. Obesity is defined as a body weight more than 20% in excess of the ideal body weight, frequently resulting in a significant impairment of health. It is associated with an increased risk for cardiovascular disease, hypertension, diabetes mellitus type II, hyperlipidaemia and an increased mortality rate. Besides severe risks of illness, individuals suffering from obesity are often isolated socially.

Obesity is influenced by genetic, metabolic, biochemical, psychological, and behavioral factors and can be caused by different reasons such as non-insulin dependent diabetes, increase in triglycerides, increase in carbohydrate bound energy and low energy expenditure. As such, it is a complex disorder that must be addressed on several fronts to achieve lasting positive clinical outcome. Since obesity is not to be considered as a single disorder but as a heterogeneous group of conditions with (potential) multiple causes, it is also characterized by elevated fasting plasma Insulin and an exaggerated insulin response to oral glucose intake (Kolterman O. G. et al., (1980) J. Clin. Invest 65: 1272-1284). A clear involvement of obesity in type II diabetes mellitus can be confirmed (Kopelman P. G., (2000) Nature 404: 635-643).

Hyperlipidemia and elevation of free fatty acids correlate clearly with the metabolic syndrome, which is defined as the linkage between several diseases, including obesity an insulin resistance. This often occurs in the same patients and is a major risk factor for development of type II diabetes and cardiovascular disease. It was suggested that the control of lipid levels and glucose levels is required to treat type II diabetes, heart disease, and other occurences of metabolic syndrome (see, for example, Santomauro A. T. et al., (1999) Diabetes, 48: 1836-1841 and Lakka H. M. et al., (2002) JAMA 288: 2709-2716).

Pancreatic beta-cells secrete insulin in response to blood glucose levels. Insulin amongst other hormones plays a key role in the regulation of the fuel metabolism. Insulin leads to the storage of glycogen and triglycerides and to the synthesis of proteins. The entry of glucose into muscles and adipose cells is stimulated by insulin. In patients who suffer from diabetes mellitus type I or latent autoimmue diabetes in adults (LADA) beta-cells are being destroyed due to autoimmune attack (Pozzilli & Di Mario, (2001) Diabetes Care. 8:1460-1467). The amount of insulin produced by the remaining pancreatic islet cells is too low, resulting in elevated blood glucose levels (hyperglycemia). In diabetes type II liver and muscle cells loose their ability to respond to normal blood insulin levels (insulin resistance). High blood glucose levels (and also high blood lipid levels) in turn lead to an impairment of beta-cell function and to an increase in beta-cell apoptosis.

Diabetes is a very disabling disease, because today's common anti-diabetic drugs do not control blood sugar levels well enough to completely prevent the occurrence of high and low blood sugar levels. Out of range blood sugar levels are toxic and cause long-term complications like for example renopathy, retinopathy, neuropathy and peripheral vascular disease. There is also a host of related conditions, such as obesity, hypertension, heart disease and hyperlipidemia, for which persons with diabetes are substantially at risk.

Apart from the impaired quality of life for the patients, the treatment of diabetes and its long-term complications presents an enormous financial burden to our healthcare systems with rising tendency. Thus, for the treatment of, type I and type II diabetes as well as for latent autoimmune diabetes in adults (LADA) there is a strong need in the art to identify factors that induce regeneration of pancreatic insulin producing beta-cells. These factors could restore normal function of the endocrine pancreas once its function is impaired or event could prevent the development or progression of diabetes type I, diabetes type II, or LADA.

The concept of metabolic syndrome (syndrome x, insulin-resistance syndrome, deadly quartet) was first described 1966 by Camus and reintroduced 1988 by Reaven (Camus J. P., (1966) Rev Rhum Mal Osteoartic 33: 10-14; Reaven G. M. et al., (1988) Diabetes, 37: 1595-1607). Today metabolic syndrome is commonly defined as clustering of cardiovascular risk factors like hypertension, abdominal obesity, high blood levels of triglycerides and fasting glucose as well as low blood levels of HDL cholesterol. Insulin resistance greatly increases the risk of developing the metabolic syndrome (Reaven G., (2002) Circulation 106: 286-288). The metabolic syndrome often precedes the development of type 2 diabetes and cardiovascular disease (Lakka H. M. et al., 2002, supra).

The molecular factors regulating food intake and body weight balance are incompletely understood. Even if several candidate genes have been described which are supposed to influence the homeostatic system(s) that regulate body mass/weight, like leptin or the peroxisome proliferator-activated receptor-gamma co-activator, the distinct molecular mechanisms and/or molecules influencing obesity or body weight/body mass regulations are not known. In addition, several single-gene mutations resulting in obesity have been described in mice, implicating genetic factors in the etiology of obesity. (Friedman J. M. and Leibel R. L., (1990) Cell 69: 217-220). In the ob mouse a single gene mutation (obese) results in profound obesity, which is accompanied by diabetes (Friedman J. M. et. al., (1991) Genomics 11: 1054-1062).

Therefore, the technical problem underlying the present invention was to provide for means and methods for modulating (pathological) metabolic conditions influencing body-weight regulation and/or energy homeostatic circuits. The solution to said technical problem is achieved by providing the embodiments characterized in the claims.

Accordingly, the present invention relates to genes with novel functions in body-weight regulation, energy homeostasis, metabolism, and obesity. The present invention discloses specific genes involved in the regulation of body-weight, energy homeostasis, metabolism, and obesity, as well as related diseases such as diabetes mellitus, eating disorder, cachexia, hypertension, coronary heart disease, hypercholesterolemia (dyslipidemia), gallstones, and liver fibrosis. In particular, the present invention describes the human PRL-1 homologous genes as being involved in those conditions mentioned above.

So far, it has not been described that a protein of the invention or a homologous protein is involved in the regulation of energy homeostasis and body-weight regulation and related disorders, and thus, no functions in metabolic diseases and dysfunctions and other diseases as listed above have been discussed.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies that are reported in the publications which might be used in connection with the invention. Nothing herein is to be construed as an admission that the invention is not entitled to antedate such disclosure.

The present invention discloses that PRL-1 (GadFly Accession Number CG4993) homologous proteins such as Prl-1, Prl-2 or Prl-3 (herein referred to as “proteins of the invention” or “a protein of the invention”) are regulating the energy homeostasis and fat metabolism, especially the metabolism and storage of triglycerides, and polynucleotides, which identify and encode the proteins disclosed in this invention. The invention also relates to vectors, host cells, and recombinant methods for producing the polypeptides and polynucleotides of the invention. The invention also relates to the use of these compounds and effectors/modulators thereof, e.g. antibodies, biologically active nucleic acids, such as antisense molecules, RNAi molecules or ribozymes, aptamers, peptides or low-molecular weight organic compounds recognizing said polynulceotides or polypeptides, in the diagnosis, study, prevention, and treatment of metabolic diseases or dysfunctions, including obesity, diabetes mellitus and/or metabolic syndrome, as well as related disorders such as eating disorder, cachexia, hypertension, coronary heart disease, hypercholesterolemia, dyslipidemia, osteoarthritis, gallstones, or liver fibrosis.

Protein tyrosine phosphorylation and dephosphorylation, catalyzed by protein tyrosine kinases (PTKs) and protein tyrosine phosphatases (PTPs), respectively, are -key switches in many important eukaryotic cellular signaling pathways. The actions of PTKs and PTPases are in a state of dynamic equilibrium that determines the status of cellular protein tyrosine phosphorylation and plays a crucial role in the regulation of cell proliferation, differentiation, viability and functional activation.

Protein tyrosine phosphatases (PTPs) form a large family of enzymes that serve as key regulatory components in signal transduction pathways by removing phosphate groups from proteins. Inappropriate regulation of PTP activity leads to wrong levels of tyrosine phosphorylation, which contributes to the development of many human diseases. For example, elevated expression of the PRL family of phosphatases such as, but not limited to, PRL PRL-2, PRL-3, and the like, has been shown to have oncogenic effects in several experimental systems and may play a causitive role in human malignancies. The PRL phosphatases are also sometimes termed PTP (CAAX) or PTP4A The PTPs, which hydrolyze the phosphate monoesters of tyrosine residues, all share a common active site motif and are classified into 3 groups. These include the receptor-like PTPs, the intracellular PTPs, and the dual-specificity PTPs, which can dephosphorylate at serine and threonine residues as well as at tyrosines.

As a member of a fourth class, a PTP from regenerating rat liver was described as PRL-I (phosphatase of regenerating liver-1) by Diamond et al. (Diamond R. H. et al. (1994) Mol Cell Biol. 14: 3752-3762). The gene, which they designated Prl-1, was one of many immediate-early genes and expressed mainly in the nucleus. Prl-1 dephosporylates ATF-7, a basic leucine zipper closely related to members of the ATF/CREB family, in vitro (Peters C. S. et al., (2001) J. Biol Chem 276: 13718-13726).

By using an in vitro prenylation screen, Cates et al. isolated two human cDNAs encoding Prl-1 homologs, designated PTP(CAAX1) and PTP(CAAX2) (Prl-2), that are farnesylated in vitro by mammalian farnesyl protein transferase. Overexpression of these PTPs in epithelial cells caused a transformed phenotype in cultured cells and tumor growth in nude mice (Cates C. A. et al., (1996) Cancer Lett 110: 49-55). Matter et al. identified a cDNA encoding PTP4A3, which they termed Prl-3. The deduced Prl-3 protein is 76% identical to Prl-1 (PTP4A1; 601585) and 96% identical to mouse Prl-3 (Matter W. F. et. al., (2001) Biochem Biophys Res Commun. 283: 1061-1068). In adult tissues, PRLs are expressed predominantly in skeletal muscle with lower expression levels detectable in brain (PRL-1), liver (PRL-2), heart (PRL-3) and pancreas (PRL-3).

PRL-1 homologous proteins and nucleic acid molecules coding therefore are obtainable from insect or vertebrate species, e.g. mammals or birds. Particularly preferred are nucleic acids encoding the human PRL-1 homologs (in particular the human Prl-1, Prl-2, and Prl-3 isoforms).

The invention particularly relates to a nucleic acid molecule encoding a polypeptide contributing to regulating the energy homeostasis and the metabolism of triglycerides, wherein said nucleic acid molecule comprises
(a) the nucleotide sequence of Drosophila PRL-1, human PRL-1 homologs (in particular the human Prl-1, Prl-2, and Prl-3 isoforms), and/or a sequence complementary thereto,
(b) a nucleotide sequence which hybridizes at 50° C. in a solution containing 1×SSC and 0.1% SDS to a sequence of (a),
(c) a sequence corresponding to the sequences of (a) or (b) within the degeneration of the genetic code,
(d) a sequence which encodes a polypeptide which is at least 85%, preferably at least 90%, more preferably at least 95%, more preferably at least 98% and up to 99.6% identical to the amino acid sequences of the PRL-1 homologous protein, preferably of the human PRL-1 homologs (in particular the human Prl-1, Prl-2, and Prl-3 isoforms),
(e) a sequence which differs from the nucleic acid molecule of (a) to (d) by mutation and wherein said mutation causes an alteration, deletion, duplication and/or premature stop in the encoded polypeptide or
(f) a partial sequence of any of the nucleotide sequences of (a) to (e) having a length of 15-25 bases, preferably 25-35 bases, more preferably 35-50 bases and most preferably at least 50 bases.

The invention is based on the finding that PRL-1 homologous proteins and the polynucleotides encoding therefor, are involved in the regulation of triglyceride storage and therefore energy homeostasis. The invention describes the use of compositions comprising PRL-1 homologous polypeptides and polynucleotides as well as modulators/effectors thereof for the diagnosis, study, prevention, or treatment of metabolic diseases or dysfunctions, including obesity, diabetes mellitus and/or metabolic syndrome, as well as related disorders such as eating disorder, cachexia, hypertension, coronary heart disease, hypercholesterolemia, dyslipidemia, osteoarthritis, gallstones or liver fibrosis.

Accordingly, the present invention relates to genes with novel functions in body-weight regulation, energy homeostasis, metabolism, and obesity, fragments of said genes, polypeptides encoded by said genes or fragments thereof, and effectors e.g. antibodies, biologically active nucleic acids, such as antisense molecules, RNAi molecules or ribozymes, aptamers, peptides or low-molecular weight organic compounds recognizing said polynucleotides or polypeptides.

The ability to manipulate and screen the genomes of model organisms such as the fly Drosophila melanogaster provides a powerful tool to analyze biological and biochemical processes that have direct relevance to more complex vertebrate organisms due to significant evolutionary conservation of genes, cellular processes, and pathways (see, for example, Adams M. D. et al., (2000) Science 287: 2185-2195). Identification of novel gene functions in model organisms can directly contribute to the elucidation of correlative pathways in mammals (humans) and of methods of modulating them. A correlation between a pathology model (such as changes in triglyceride levels as indication for metabolic syndrome including obesity) and the modified expression of a fly gene can identify the association of the human ortholog with the particular human disease.

In one embodiment, a forward genetic screen Is performed in fly displaying a mutant phenotype due to misexpression of a known gene (see, St Johnston D., (2002) Nat Rev Genet 3: 176-188; Rorth P., (1996) Proc Natl Acad Sci USA 93: 12418-12422). In this invention, we have used a genetic screen to identify mutations of PRL-1 homologous genes that cause changes in the body weight which is reflected by a significant change of triglyceride levels.

Obese people mainly show a significant increase in the content of triglycerides. Triglycerides are the most efficient storage for energy in cells. In order to isolate genes with a function in energy homeostasis, several thousand proprietary and publicly available EP-lines were tested for their triglyceride content after a prolonged feeding period (see Examples and Figures for more detail). Lines with significantly changed triglyceride content were selected as positive candidates for further analysis. The change of triglyceride content due to the loss of a gene function suggests gene activities in energy homeostasis in a dose dependent manner that control the amount of energy stored as triglycerides.

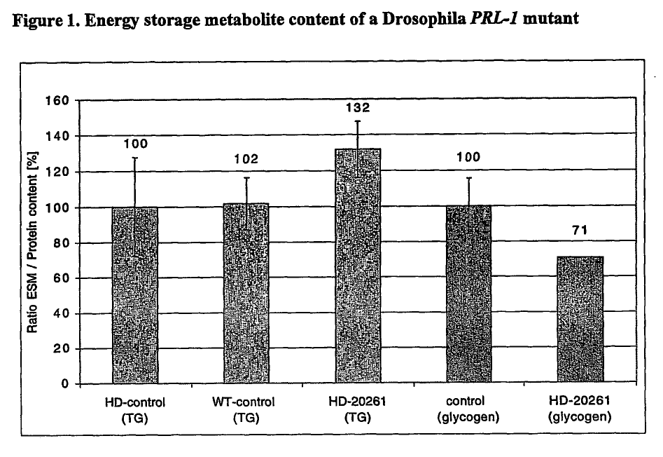

In this invention, the content of triglycerides of a pool of flies with the same genotype was analyzed after feeding for six days using a triglyceride assay. Male flies homozygous for the integration of vectors for Drosophila line HD-EP (2)20261 were analyzed in assays measuring the triglyceride and glycogen contents of these flies, illustrated in more detail in the Examples section. The results of the triglyceride and glycogen content analysis are shown in FIG. 1.

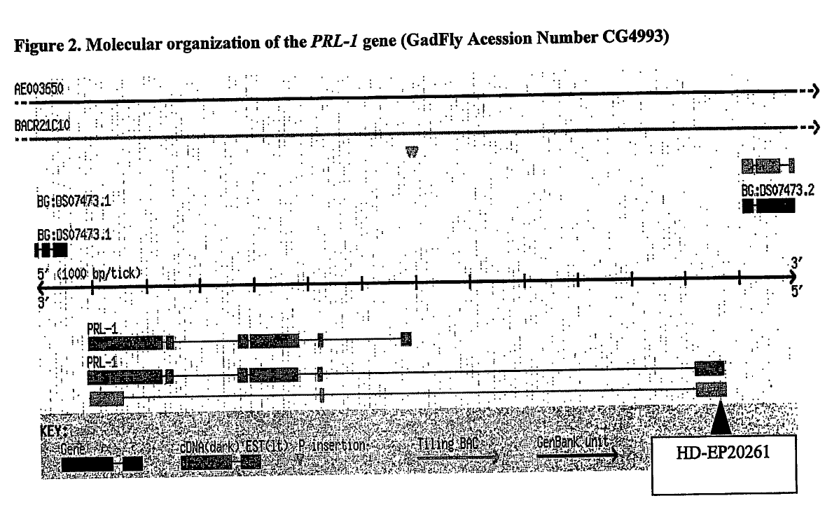

Genomic DNA sequences were isolated that are localized to the EP vector (herein HD-EP(2)20261) integration. Using those isolated genomic sequences public databases like Berkeley Drosophila Genome Project (GadFly; see also FlyBase (1999) Nucleic Acids Research 27: 85-88) were screened thereby identifying the integration site of the vectors, and the corresponding gene, described in more detail in the Examples section. The molecular organization of the gene is shown in FIG. 2.

The Drosophila PRL-1 gene and protein encoded thereby with functions in the regulation of triglyceride metabolism was further analysed in publicly available sequence databases (see Examples for more detail) and mammalian homologs were identified.

The function of the mammalian homologs in energy homeostasis was further validated in this invention by analyzing the expression of the transcripts in different tissues and by analyzing the role in adipocyte differentiation. In one emdociment of the invention, we clearly show that the mammalian homologues of the PRL-1 gene play a central role in energy homeostasis (see Examples for more detail).

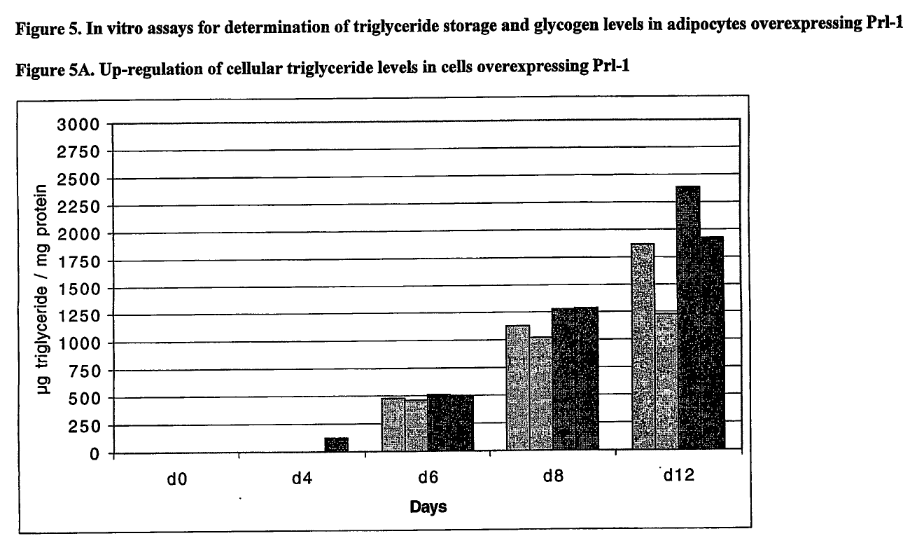

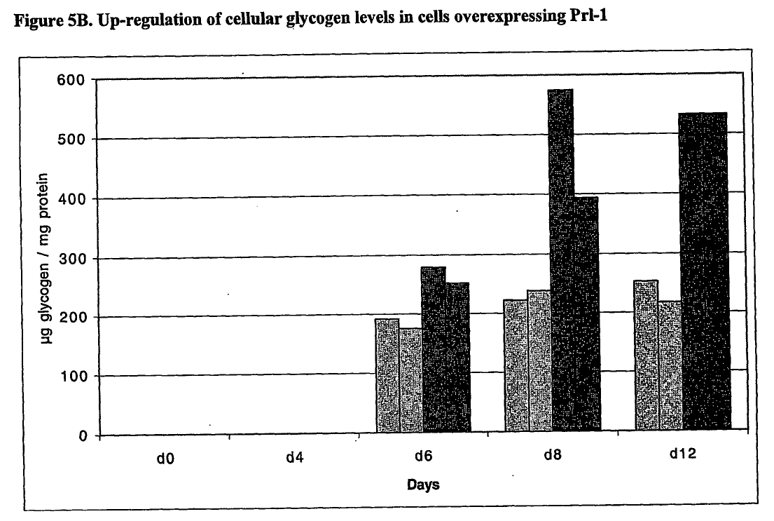

Using a model of adipocyte differentiation, we found that in Prl-1 overexpressing cells triglyceride levels were significantly increased on day 12 of differentiation (see FIG. 5A). This increase is also seen in control experiments with known regulators of adipogenesis, such as for example PPAR gamma-1.

In human SGBS cells Prl-1 over-expression led to a significant increase in free fatty acid and glucose uptake into differentiated adipocytes. Free fatty acids and glucose can be metabolized and stored by adipocytes as triglyceride or glycogen.

Glycogen levels in cells are more variable than triglyceride levels because the turnover of glycogen is higher. Glucose is taken up by the cells rapidly and stored in the form of glycogen. This energy storage is then used as a first quick response to the metabolic demands of the cell. On day 12 of differentiation of adipocytes, intracellular glycogen levels are significantly increased in cells overexpressing Prl-1 (see FIG. 5B).

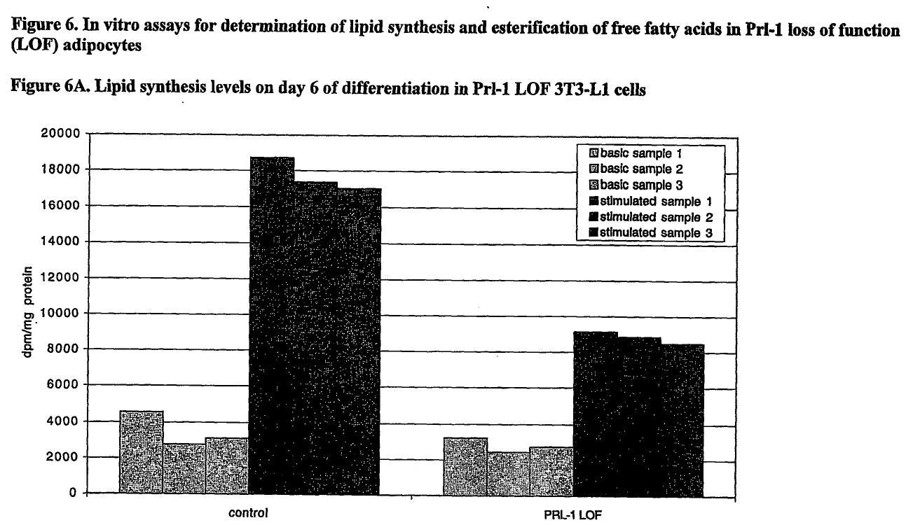

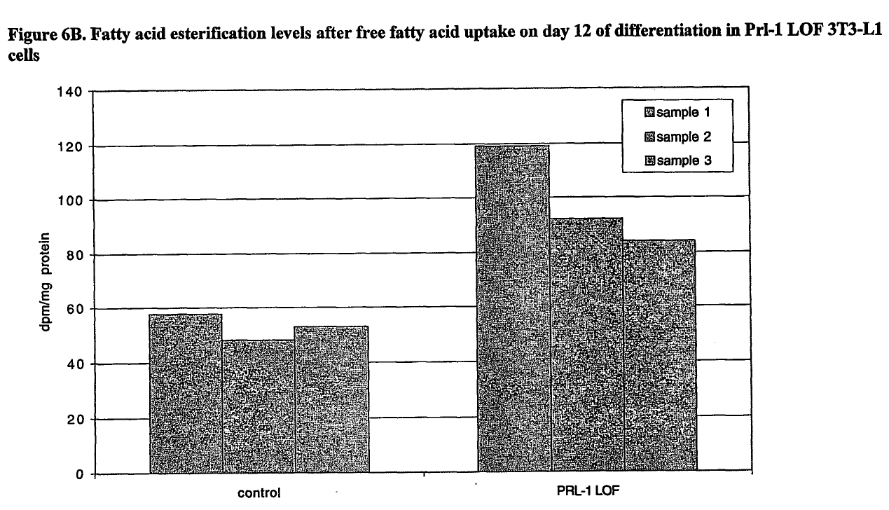

Further, we found that In Prl-1 loss of function (LOF) cells insulin stimulated lipid synthesis levels were significantly decreased on day 6 of differentiation, when compared with controls (see FIG. 6A). On day 12 of adipocyte differentiation, fatty acid esterification levels are up-regulated after uptake of free fatty acids in Prl-1 LOF cells, when compared with control cells (see FIG. 6B).

These findings indicate that the Prl-1 affects central metabolic pathways in the cell.

Microarrays are analytical tools routinely used in bioanalysis. A microarray has molecules distributed over, and stably associated with, the surface of a solid support. The term “microarray” refers to an arrangement of a plurality of polynucleotides, polypeptides, antibodies, or other chemical compounds on a substrate. Microarrays of polypeptides, polynucleotides, and/or antibodies have been developed and find use in a variety of applications, such as monitoring gene expression, drug discovery, gene sequencing, gene mapping, bacterial identification, and combinatorial chemistry. One area in particular in which microarrays find use is in gene expression analysis (see Example 7). Array technology can be used to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Microarrays may be prepared, used, and analyzed using methods known in the art (see for example, Brennan T. M., (1995) U.S. Pat. No. 5,474,796; Schena M. et al., (1996) Proc. Natl. Acad. Sci. USA 93: 10614-10619; Baldeschwieler et al., (1995) PCT application WO9525116; Shalon T. D. and Brown P. O., (1995) PCT application WO9535505; Heller R. A. et al., (1997) Proc. Natl. Acad. Sci. USA 94: 2150-2155; Heller M. J. and Tu E., (1997) U.S. Pat. No. 5,605,662). Various types of microarrays are well known and thoroughly described in Schena M., ed. (1999); DNA Microarrays: A Practical Approach, Oxford University Press, London.

Oligonucleotides or longer fragments derived from any of the polynucleotides described herein may be used as elements on a microarray. The microarray can be used In transcript imaging techniques, which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient For example, therapeutic agents, which are highly effective and display the fewest side effects may be selected for a patient based on his/her pharmacogenomic profile.

As determined by microarray analysis, protein tyrosine phosphatase type IVA, member 1 (PRL-1) shows differential expression in human primary adipocytes. Thus, PRL-1 is a strong candidate for the manufacture of a pharmaceutical composition and a medicament for the treatment of conditions related to human metabolism, such as obesity, diabetes, and/or metabolic syndrome.

The invention also encompasses polynucleotides that encode the proteins of the invention and homologous proteins. Accordingly, any nucleic acid sequence, which encodes the amino acid sequences of the proteins of the invention and homologous proteins, can be used to generate recombinant molecules that express the proteins of the invention and homologous proteins. In a particular embodiment, the invention encompasses a nucleic acid encoding Drosophila PRL-1 or human PRL-1 homologs; referred to herein as the proteins of the invention. It will be appreciated by those skilled in the art that as a result of the degeneracy of the genetic code, a multitude of nucleotide sequences encoding the proteins, some bearing minimal homology to the nucleotide sequences of any known and naturally occurring gene, may be produced. The invention contemplates each and every possible variation of nucleotide sequence that can be made by selecting combinations based on possible codon choices.

Also encompassed by the invention are polynucleotide sequences that are capable of hybridizing to the claimed nucleotide sequences, and in particular, those of the polynucleotide encoding the proteins of the invention, under various conditions of stringency. Hybridization conditions are based on the melting temperature (Tm) of the nucleic acid binding complex or probe, as described in Wahl G. M. et al. (1987: Methods Enzymol. 152: 399-407) and Kimmel A. R. (1987; Methods Enzymol. 152: 507-511), and may be used at a defined stringency. Preferably, hybridization under stringent conditions means that after washing for 1 h with 1×SSC and 0.1% SDS at 50° C., preferably at 55° C., more preferably at 62° C., and most preferably at 65° C., particularly for 1 h in 0.2×SSC and 0.1% SDS at 50° C., preferably at 55° C., more preferably at 62° C., and most preferably 65° C. a positive hybridization signal is observed. Altered nucleic acid sequences encoding the proteins which are encompassed by the invention include deletions, insertions or substitutions of different nucleotides resulting in a polynucleotide that encodes the same or a functionally equivalent protein.

The encoded proteins may also contain deletions, insertions or substitutions of amino acid residues, which produce a silent change and result in functionally equivalent proteins. Deliberate amino acid substitutions may be made on the basis of similarity in polarity, charge, solubility, hydrophobicity, hydrophilicity, and/or the amphipathic nature of the residues as long as the biological activity of the protein is retained. Furthermore, the invention relates to peptide fragments of the proteins or derivatives thereof such as cyclic peptides, retro-inverso peptides or peptide mimetics having a length of at least 4, preferably at least 6 and up to 50 amino acids.

Also included within the scope of the present invention are alleles of the genes encoding the proteins of the invention and homologous proteins. As used herein, an ‘allele’ or ‘allelic sequence’ is an alternative form of the gene, which may result from at least one mutation in the nucleic acid sequence. Alleles may result in altered mRNAs or polypeptides whose structures or function may or may not be altered. Any given gene may have none, one or many allelic forms. Common mutational changes, which give rise to alleles, are generally ascribed to natural deletions, additions or substitutions of nucleotides. Each of these types of changes may occur alone or in combination with the others, one or more times in a given sequence.

The nucleic acid sequences encoding the proteins of the invention and homologous proteins may be extended utilizing a partial nucleotide sequence and employing various methods known in the art to detect upstream sequences such as promoters and regulatory elements.

In order to express a biologically active protein, the nucleotide sequences encoding the proteins or functional equivalents, may be inserted into appropriate expression vectors, i.e., a vector which contains the necessary elements for the transcription and translation of the inserted coding sequence. Methods, which are well known to those skilled in the art, may be used to construct expression vectors containing sequences encoding the proteins and the appropriate transcriptional and translational control elements. Regulatory elements include for example a promoter, an initiation codon, a stop codon, a mRNA stability regulatory element, and a polyadenylation signal. Expression of a polynucleotide can be assured by (i) constitutive promoters such as the Cytomegalovirus (CMV) promoter/enhancer region, (ii) tissue specific promoters such as the insulin promoter (see, Soria B. et al., (2000) Diabetes 49: 157-162), SOX2 gene promoter (see Li M. et al., (1998) Curr. Biol. 8: 971-974), Msi-1 promoter (see Sakakibara S. and Okano H., (1997) J. Neuroscience 17: 8300-8312), alpha-cardia myosin heavy chain promotor or human atrial natriuretic factor promoter (Klug M. G. et al., (1996) J. Clin. Invest 98: 216-224; Wu J. et al., (1989) J. Biol. Chem. 264: 6472-6479) or (iii) inducible promoters such as the tetracycline inducible system. Expression vectors can also contain a selection agent or marker gene that confers antibiotic resistance such as the neomycin, hygromycin or puromycin resistance genes. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y. and Ausubel, F. M. et al. (1989) Current Protocols in Molecular Biology, John Wiley & Sons, New York, N.Y.

In a further embodiment of the invention, natural, modified or recombinant nucleic acid sequences encoding the proteins of the invention and homologous proteins may be ligated to a heterologous sequence to encode a fusion protein.

A variety of expression vector/host systems may be utilized to contain and express sequences encoding the proteins or fusion proteins. These include, but are not limited to, micro-organisms such as bacteria transformed with recombinant bacteriophage, plasmid or cosmid DNA expression vectors; yeast transformed with yeast expression vectors; insect cell systems infected with virus expression vectors (e.g., baculovirus, adenovirus, adeno-associated virus, lentiverus, retrovirus); plant cell systems transformed with virus expression vectors (e.g., cauliflower mosaic virus, CaMV; tobacco mosaic virus, TMV) or with bacterial expression vectors (e.g., Ti or PBR322 plasmids); or animal cell systems.

The presence of polynucleotide sequences of the invention in a sample can be detected by DNA-DNA or DNA-RNA hybridization and/or amplification using probes or portions or fragments of said polynucleotides. Nucleic acid amplification based assays involve the use of oligonucleotides or oligomers based on the sequences specific for the gene to detect transformants containing DNA or RNA encoding the corresponding protein. As used herein ‘oligonucleotides’ or ‘oligomers’ refer to a nucleic acid sequence of at least about 10 nucleotides and as many as about 60 nucleotides, preferably about 15 to 30 nucleotides, and more preferably about 20-25 nucleotides, which can be used as a probe or amplimer.

A wide variety of labels and conjugation techniques are known by those skilled in the art and may be used in various nucleic acid and amino acid assays. Means for producing labeled hybridization or PCR probes for detecting polynucleotide sequences include oligo-labeling, nick translation, end-labeling of RNA probes, PCR amplification using a labeled nucleotide, or enzymatic synthesis. These procedures may be conducted using a variety of commercially available kits (Pharmacia & Upjohn, (Kalamazoo, Mich.); Promega (Madison Wis.); and U.S. Biochemical Corp., (Cleveland, Ohio).

The presence of proteins of the invention in a sample can be determined by immunological methods or activity measurement. A variety of protocols for detecting and measuring the expression of proteins, using either polyclonal or monoclonal antibodies specific for the protein or reagents for determining protein activity are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on the protein is preferred, but a competitive binding assay may be employed. These and other assays are described, among other places, in Hampton, R et al. (1990; Serological Methods, a Laboratory Manual, APS Press, St Paul, Minn.) and Maddox, D. E. et al. (1983; J. Exp. Med. 158: 1211-1226).

Suitable reporter molecules or labels, which may be used, include radionuclides, enzymes, fluorescent, chemiluminescent or chromogenic agents as well as substrates, co-factors, inhibitors, magnetic particles, and the like.

Host cells transformed with nucleotide sequences encoding a protein of the invention may be cultured under conditions suitable for the expression and recovery of said protein from cell culture. The protein produced by a recombinant cell may be secreted or contained intracellularly depending on the sequence or/and the vector used. As will be understood by those of skill in the art, expression vectors containing polynucleotides, which encode the protein may be designed to contain signal sequences, which direct secretion of the protein through a prokaryotic or eukaryotic cell membrane. Other recombinant constructions may be used to join sequences encoding the protein to nucleotide sequence encoding a polypeptide domain, which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAG extension/affinity purification system (Immunex Corp., Seattle, Wash.) The inclusion of cleavable linker sequences such as those specific for Factor XA or Enterokinase (Invitrogen, San Diego, Calif.) between the purification domain and the desired protein may be used to facilitate purification.

Diagnostics and Therapeutics

The data disclosed in this invention show that the nucleic acids and proteins of the invention and modulator/effector molecules thereof are useful in diagnostic and therapeutic applications implicated, for example, but not limited to, in metabolic diseases or dysfunctions including obesity, diabetes mellitus, and/or metabolic syndrome as well as related disorders such as eating disorder, cachexia, hypertension, coronary heart disease, hypercholesterolemia, dyslipidemia, osteoarthritis, gallstones, or liver fibrosis. Hence, diagnostic and therapeutic uses for the proteins of the invention nucleic acids and proteins of the invention are, for example but not limited to, the following: (i) protein therapy (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention and effectors thereof are useful in diagnostic and therapeutic applications implicated in various applications as described below. For example, but not limited to, cDNAs encoding the proteins of the invention and particularly their human homologues may be useful in gene therapy, and the proteins of the invention and particularly their human homologues may be useful when administered to a subject in need thereof. By way of non-limiting example, the compositions of the present invention will have efficacy for treatment of patients suffering from, for example, but not limited to, in metabolic disorders as described above.

The nucleic acids of the invention or fragments thereof, may further be useful in diagnostic applications, wherein the presence or amount of the nucleic acids or the proteins are to be assessed. Further antibodies that bind immunospecifically to the substances of the invention may be used in therapeutic or diagnostic methods.

For example, in one aspect, antibodies, which are specific for the proteins of the invention and homologous proteins, may be used directly as an effector, e.g. an antagonist or indirectly as a targeting or delivery mechanism for bringing a pharmaceutical agent to cells or tissue which express the protein. The antibodies may be generated using methods that are well known in the art. Such antibodies may include, but are not limited to, polyclonal, monoclonal, chimeric single chain, Fab fragments, and fragments produced by a Fab expression library. Neutralising antibodies, (i.e., those which inhibit dimer formation) are especially preferred for therapeutic use.

For the production of antibodies, various hosts including goats, rabbits, rats, mice, humans, and others, may be immunized by injection with the protein or any fragment or oligopeptide thereof which has immunogenic properties. Depending on the host species, various adjuvants may be used to increase immunological response. It is preferred that the peptides, fragments or oligopeptides used to induce antibodies to the protein have an amino acid sequence consisting of at least five amino adds, and more preferably at least 10 amino acids.

Monoclonal antibodies to the proteins may be prepared using any technique that provides for the production of antibody molecules by continuous cell lines in culture. These include, but are not limited to, the hybridoma technique, the human B-cell hybridoma technique, and the EBV-hybridoma technique (Köhler G. and Milstein C., (1975) Nature 256: 495-497; Kozbor D. et al., (1985) J. Immunol. Methods 81: 31-42; Cote R. J. et al., (1983) Proc. Natl. Acad. Sci. USA 80: 2026-2030; Cole S. P. et al., (1984) Mol. Cell Biochem. 62: 109-120).

In addition, techniques developed for the production of ‘chimeric antibodies’, the splicing of mouse antibody genes to human antibody genes to obtain a molecule with appropriate antigen specificity and biological activity can be used (Morrison S. L. et al., (1984) Proc. Natl. Acad. Sci. USA 81: 6851-6855; Neuberger M. S. et al., (1984) Nature 312: 604-608; Takeda S. et al., (1985) Nature 314: 452-454). Alternatively, techniques described for the production of single chain antibodies may be adapted, using methods known in the art, to produce single chain antibodies specific for the proteins of the invention and homologous proteins. Antibodies with related specificity, but of distinct idiotypic composition, may be generated by chain shuffling from random combinatorial immunoglobulin libraries (Kang A. S. et al., (1991) Proc. Natl. Acad. Sci. USA 88: 11120-11123). Antibodies may also be produced by inducing in vivo production in the lymphocyte population or by screening recombinant immunoglobulin libraries or panels of highly specific binding reagents as disclosed in the literature (Orlandi R. et al., (1989) Proc. Natl. Acad. Sci. USA 86: 3833-3837; Winter G. and Milstein C., (1991) Nature 349: 293-299).

Antibody fragments which contain specific binding sites for the proteins may also be generated. For example, such fragments include, but are not limited to, the F(ab′)₂ fragments which can be produced by Pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse W. D. et al., (1989) Science 246: 1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between the protein and its specific antibody. A two-site, monoclonal-based immunoassay utilizing monoclonal antibodies reacive to two non-interfering protein epitopes are preferred, but a competitive binding assay may also be employed (Maddox, supra).

In another embodiment of the invention, the polynucleotides of the invention or fragments thereof or nucleic acid modulator/effector molecules such as aptamers, antisense molecules, RNAi molecules or ribozymes may be used for therapeutic purposes. In one aspect, aptamers, i.e. nucleic acid molecules, which are capable of binding to a Prl protein and modulating its activity, may be generated by a screening and selection procedure involving the use of combinatorial nucleic acid libraries.

In a further aspect, antisense molecules may be used in situations in which it would be desirable to block the transcription of the mRNA In particular, cells may be transformed with sequences complementary to polynucleotides encoding the proteins of the invention and homologous proteins. Thus, antisense molecules may be used to modulate protein activity or to achieve regulation of gene function. Such technology is now well known in the art, and sense or antisense oligomers or larger fragments, can be designed from various locations along the coding or control regions of sequences encoding the proteins. Expression vectors derived from retroviruses, adenovirus, herpes or vaccinia viruses or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the genes encoding the proteins of the invention and homologous proteins. These techniques are described both in Sambrook et al. (supra) and in Ausubel et al. (supra). Genes encoding the proteins of the invention and homologous proteins can be turned off by transforming a cell or tissue with expression vectors, which express high levels of polynucleotides that encode the proteins of the Invention and homologous proteins or fragments thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, e.g. DNA, RNA or nucleic acid analogues such as PNA, to the control regions of the genes encoding the proteins of the invention and homologous proteins, i.e., the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors or regulatory molecules. Recent therapeutic advances using triplex DNA have been described in the literature (Gee J. E. et al., (1994) Gene 149: 109-114; Huber, B. E. and Carr B. I., Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples, which may be used, include engineered hammerhead motif ribozyme molecules that can be specifically and efficiently catalyze endonucleolytic cleavage of sequences encoding the proteins of the invention and homologous proteins. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Nucleic acid effector molecules, e.g. antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences. Such DNA sequences may be incorporated into a variety of vectors with suitable RNA polymerase promoters such as T7 or SP6. Alternatively, these cDNA constructs that synthesize antisense RNA constitutively or inducibly can be introduced into cell lines, cells or tissues. RNA molecules may be modified to increase intracellular stability and half-life. Possible modifications include, but are not limited to, the addition of flanking sequences at the 5′ and/or 3′ ends of the molecule or modifications in the nucleobase, sugar and/or phosphate moieties, e.g. the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition; in conjunction with a pharmaceutically acceptable carrier, for any of the therapeutic effects discussed above. Such pharmaceutical compositions may consist of the nucleic acids and the proteins of the invention and homologous nucleic acids or proteins, antibodies to the proteins of the invention and homologous proteins, mimetics, agonists, antagonists or Inhibitors of the proteins of the invention and homologous proteins or nucleic acids. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone or in combination with other agents, drugs or hormones. The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual or rectal means.

In addition to the active ingredients, these pharmaceutical compositions may contain suitable pharmaceutically-acceptable carriers comprising excipients and auxiliaries, which facilitate processing of the active compounds into preparations, which can be used pharmaceutically. Further details on techniques for formulation and administration may be found in the latest edition of Remington's Pharmaceutical Sciences (Maack Publishing Co., Easton, Pa.).

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective dose can be estimated initially either in cell culture assays, e.g., of preadipocyte cell lines or in animal models, usually mice, rabbits, dogs or pigs. The animal model may also be used to determine the appropriate concentration range and route of administration. Such information can then be used to determine useful doses and routes for administration in humans. A therapeutically effective dose refers to that amount of active ingredient, for example the nucleic acids or the proteins of the invention or fragments thereof, or antibodies of the proteins of the invention and homologous proteins, which is sufficient for treating a specific condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind to the proteins may be used for the diagnosis of conditions or diseases characterized by or associated with over- or underexpression of the proteins of the invention and homologous proteins or in assays to monitor patients being treated with the proteins of the invention and homologous proteins, or effectors thereof, e.g. agonists, antagonists, or inhibitors. Diagnostic assays include methods which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules which are known in the art may be used several of which are described above.

A variety of protocols including ELISA, RIA, and FACS for measuring proteins are known in the art and provide a basis for diagnosing altered or abnormal levels of gene expression. Normal or standard values for gene expression are established by combining body fluids or cell extracts taken from normal mammalian subjects, preferably human, with antibodies to the protein under conditions suitable for complex formation. The amount of standard complex formation may be quantified by various methods, but preferably by photometric means. Quantities of protein expressed in control and disease, samples e.g. from biopsied tissues are compared with the standard values. Deviation between standard and subject values establishes the parameters for diagnosing disease.

In another embodiment of the invention, the polynucleotides specific for the proteins of the invention and homologous proteins may be used for diagnostic purposes. The polynucleotides, which may be used, include oligonucleotide sequences, antisense RNA and DNA molecules, and PNAs. The polynucleotides may be used to detect and quantitate gene expression in biopsied tissues in which gene expression may be correlated with disease. The diagnostic assay may be used to distinguish between absence, presence, and excess gene expression, and to monitor regulation of protein levels during therapeutic intervention.

In one aspect, hybridization with probes which are capable of detecting polynucleotide sequences, including genomic sequences, encoding the proteins of the invention and homologous proteins or closely related molecules, may be used to identify nucleic acid sequences which encode the respective protein. The hybridization probes of the subject invention may be DNA or RNA and are preferably derived from the nucleotide sequence of the polynucleotide encoding the proteins of the invention or from a genomic sequence including promoter, enhancer elements, and introns of the naturally occurring gene. Hybridization probes may be labeled by a variety of reporter groups, for example, radionuclides such as ³²P or ³⁵S or enzymatic labels, such as alkaline phosphatase coupled to the probe via avidin/biotin coupling systems, and the like.

Polynucleotide sequences specific for the proteins of the invention and homologous nucleic acids may be used for the diagnosis of conditions or diseases, which are associated with the expression of the proteins. Examples of such conditions or diseases include, but are not limited to, metabolic diseases and disorders, including obesity and diabetes. Polynucleotide sequences specific for the proteins of the invention and homologous proteins may also be used to monitor the progress of patients receiving treatment for metabolic diseases and disorders, including obesity and diabetes. The polynucleotide sequences may be used qualitative or quantitative assays, e.g. in Southern or Northern analysis, dot blot or other membrane-based technologies; in PCR technologies; or in dip stick, pin, ELISA or chip assays utilizing fluids or tissues from patient biopsies to detect altered gene expression.

In a particular aspect, the nucleotide sequences specific for the proteins of the invention and homologous nucleic acids may be useful in assays that detect activation or induction of various metabolic diseases or dysfunctions, for example, obesity, diabetes mellitus, eating disorder, cachexia, hypertension, coronary heart disease, hypercholesterolemia (dyslipidemia), gallstones, or liver fibrosis. The nucleotide sequences may be labeled by standard methods, and added to a fluid or tissue sample from a patient under conditions suitable for the formation of hybridization complexes. After a suitable incubation period, the sample is washed and the signal is quantitated and compared with a standard value. The presence of altered levels of nucleotide sequences encoding the proteins of the invention and homologous proteins in the sample indicates the presence of the associated disease. Such assays may also be used to evaluate the efficacy of a particular therapeutic treatment regimen in animal studies, in clinical trials or in monitoring the treatment of an individual patient.

In order to provide a basis for the diagnosis of a disease associated with expression of the proteins of the invention and homologous proteins, a normal or standard profile for expression is established. This may be accomplished by combining body fluids or cell extracts taken from normal subjects, either animal or human, with a sequence or a fragment thereof, which is specific for the nucleic acids encoding the proteins of the invention and homologous nucleic acids, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridization assays may be repeated on a regular basis to evaluate whether the level of expression in the patient begins to approximate that, which is observed in the normal patent. The results obtained from successive assays may be used to show the efficacy of treatment over a period ranging from several days to months.

With respect to metabolic diseases such as described above the presence of an unusual amount of transcript in biopsied tissue from an individual may indicate a predisposition for the development of the disease or may provide a means for detecting the disease prior to the appearance of actual clinical symptoms. A more definitive diagnosis of this type may allow health professionals to employ preventative measures or aggressive treatment earlier thereby preventing the development or further progression of the metabolic diseases and disorders.

Additional diagnostic uses for oligonucleotides designed from the sequences encoding the proteins of the invention and homologous proteins may involve the use of PCR. Such oligomers may be chemically synthesized, generated enzymatically or produced from a recombinant source. Oligomers will preferably consist of two nucleotide sequences, one with sense orientation (5prime.fwdarw.3prime) and another with antisense (3prime.rarw.5prime), employed under optimized conditions for identification of a specific gene or condition. The same two oligomers, nested sets of oligomers or even a degenerate pool of oligomers may be employed under less stringent conditions for detection and/or quantification of closely related DNA or RNA sequences.

In another embodiment of the invention, the nucleic acid sequences may also be used to generate hybridization probes, which are useful for mapping the naturally occurring genomic sequence. The sequences may be mapped to a particular chromosome or to a specific region of the chromosome using well known techniques. Such techniques include FISH, FACS or artificial chromosome constructions, such as yeast artificial chromosomes, bacterial artificial chromosomes, bacterial P1 constructions or single chromosome cDNA libraries as reviewed in Price C. M., (1993) Blood Rev. 7: 127-134, and Trask B. J., (1991) Trends Genet. 7: 149-154. FISH (as described in Verma R. S. and Babu A., (1989) Human Chromosomes: A Manual of Basic Techniques, Pergamon Press, New York, N.Y.). The results may be correlated with other physical chromosome mapping techniques and genetic map data. Examples of genetic map data can be found in the 1994 Genome Issue of Science (265: 1981f). Correlation between the location of the gene encoding the proteins of the invention on a physical chromosomal map and a specific disease or predisposition to a specific disease, may help to delimit the region of DNA associated with that genetic disease.

The nucleotide sequences of the subject invention may be used to detect differences in gene sequences between normal, carrier or affected individuals. An analysis of polymorphisms, e.g. single nucleotide polymorphisms may be carried out. Further, in situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (Gatti R. A. et al., (1988) Nature 336: 577-580), any sequences mapping to that area may represent associated or regulatory genes for further investigation. The nucleotide sequences of the subject invention may also be used to detect differences in the chromosomal location due to translocation, inversion, etc. among normal, carrier or affected individuals.

In another embodiment of the invention, the proteins of the invention, their catalytic or immunogenic fragments or oligopeptides thereof, an In vitro model, a genetically altered cell or animal, can be used for screening libraries of compounds in any of a variety of drug screening techniques. One can identify effectors, e.g. receptors, enzymes, proteins, ligands, or substrates that bind to, modulate or mimic the action of one or more of the proteins of the invention. The protein or fragment thereof employed in such screening may be free in solution, affixed to a solid support, borne on a cell surface, or located intracellulary. The formation of binding complexes, between the proteins of the invention and the agent tested, may be measured. Agents can also be identified, which, either directly or Indirectly, influence the activity of the protein of the invention. For example the phosphatase activity of the proteins of the invention could be measured in vitro by using recombinantly expressed and purified Prl-1 or fragments thereof by making use of artificial substrates well known in the art, i.e. but not exclusively DiFMUP (Molecular Probes, Eugene, Oreg.), which are converted to fluorophores or chromophores upon dephosphorylation. Alternatively, the dephosphorylation of physiological substrates of the phosphatases could be measured by making use of any of the well known screening technologies suitable for the detection of the phosphorylation status of their physiological substrates. For example, but not exclusively, the phosphorylation status of peptides derived from their physiological substrates can be monitored by binding of phospho-side specific antibodies resulting in an increase of the polarization of the complex.

In addition activity of PRL-1 homologous proteins against their physiological substrate(s) or derivatives thereof could be measured in cell-based assays. Agents may also interfere with posttranslational modifications of the protein, such as phosphorylation and dephosphorylation, farnesylation, palmitoylation, acetylation, alkylation, ubiquitination, proteolytic processing, subcellular localization and degradation. Moreover, agents could influence the dimerization or oligomerization of the proteins of the invention or, in a heterologous manner, of the proteins of the invention with other proteins, for example, but not exclusively, docking proteins, enzymes, receptors, or translation factors. Agents could also act on the physical interaction of the proteins of this invention with other proteins, which are required for protein function, for example, but not exclusively, their downstream signaling.

Methods for determining protein-protein interaction are well known in the art. For example binding of a fluorescently labeled peptide derived from the interacting protein to the protein of the Invention, or vice versa, could be detected by a change in polarisation. In case that both binding partners, which can be either the full length proteins as well as one binding partner as the full length protein and the other just represented as a peptide are fluorescently labeled, binding could be detected by fluorescence energy transfer (FRET) from one fluorophore to the other. In addition, a variety of commercially available assay principles suitable for detection of protein-protein Interaction are well known In the art, for example but not exclusively AlphaScreen (PerkinElmer) or Scintillation Proximity Assays (SPA) by Amersham. Alternatively, the interaction of the proteins of the invention with cellular proteins could be the basis for a cell-based screening assay, in which both proteins are fluorescently labeled and interaction of both proteins is detected by analysing cotranslocation of both proteins with a cellular imaging reader, as has been developed for example, but not exclusively, by Cellomics or EvotecOAI. In all cases the two or more binding partners can be different proteins with one being the protein of the invention, or in case of dimerization and/or oligomerization the protein of the invention itself. Proteins of the invention, for which one target mechanism of interest, but not the only one, would be such protein/protein interactions are PRL-1 homologous proteins.

Of particular interest are screening assays for agents that have a low toxicity for mammalian cells. The term “agent” as used herein describes any molecule, e.g. protein or pharmaceutical, with the capability of altering or mimicking the physiological function of one or more of the proteins of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

Another technique for drug screening, which may be used, provides for high throughput screening of compounds having suitable binding affinity to the protein of interest as described in published PCT application WO84/03564. In this method, as applied to the proteins of the invention large numbers of different small test compounds, e.g. aptamers, peptides, low-molecular weight compounds etc., are provided or synthesized on a solid substrate, such as plastic pins or some other surface. The test compounds are reacted with the proteins or fragments thereof, and washed. Bound proteins are then detected by methods well known in the art. Purified proteins can also be coated directly onto plates for use in the aforementioned drug screening techniques. Alternatively, non-neutralizing antibodies can be used to capture the peptide and immobilize it on a solid support. In another embodiment, one may use competitive drug screening assays in which neutralizing antibodies capable of binding the protein specifically compete with a test compound for binding the protein. In this manner, the antibodies can be used to detect the presence of any peptide, which shares one or more antigenic determinants with the protein.

The nucleic acids encoding the protein of the invention can be used to generate transgenic animals or site-specific gene modifications in cell lines. These transgenic non-human animals are useful in the study of the function and regulation of the protein of the invention in vivo. Transgenic animals, particularly mammalian transgenic animals, can serve as a model system for the investigation of many developmental and cellular processes common to humans. A variety of non-human models of metabolic disorders can be used to test effectors/modulators of the protein of the invention. Misexpression (for example, overexpression or lack of expression) of the protein of the invention, particular feeding conditions, and/or administration of biologically active compounds can create models of metablic disorders.

In one embodiment of the invention, such assays use mouse models of insulin resistance and/or diabetes, such as mice carrying gene knockouts in the leptin pathway (for example, ob (leptin) or db (leptin receptor) mice). Such mice develop typical symptoms of diabetes, show hepatic lipid accumulation and frequently have increased plasma lipid levels (see Bruning J. C. et al., 1998, supra). Susceptible wild type mice (for example C57Bl/6) show similiar symptoms if fed a high fat diet. In addition to testing the expression of the proteins of the invention in such mouse strains (see Examples section), these mice could be used to test whether administration of a candidate effector/modulator alters for example lipid accumulation in the liver, in plasma, or adipose tissues using standard assays well known in the art, such as FPLC, colorimetric assays, blood glucose level tests, insulin tolerance tests and others.

Transgenic animals may be made through homologous recombination in non-human embryonic stem cells, where the normal locus of the gene encoding the protein of the invention is altered. Alternatively, a nucleic acid construct encoding the protein of the invention is injected into oocytes and is randomly integrated into the genome. Vectors for stable integration include plasmids, retroviruses and other animal viruses, yeast artificial chromosomes (YACs), and the like. The modified cells or animals are useful in the study of the function and regulation of the protein of the invention. For example, a series of small deletions and/or substitutions may be made in the gene that encodes the protein of the invention to determine the role of particular domains of the protein, functions in pancreatic differentiation, etc.

Furthermore, variants of the gene of the invention like specific constructs of interest include anti-sense molecules, which will block the expression of the protein of the invention, or expression of dominant negative mutations. A detectable marker, such as for example lac-Z or luciferase may be introduced in the locus of the gene of the invention, where up regulation of expression of the gene of the invention will result in an easily detected change in phenotype.

One may also provide for expression of the gene of the invention or variants thereof in cells or tissues where it is not normally expressed or at abnormal times of development. In addition, by providing expression of the protein of the invention in cells in which they are not normally produced, one can induce changes in cell behavior.

DNA constructs for homologous recombination will comprise at least portions of the gene of the invention with the desired genetic modification, and will include regions of homology to the target locus. DNA constructs for random integration do not need to contain regions of homology to mediate recombination. Conveniently, markers for positive and negative selection are included. DNA constructs for random integration will consist of the nucleic acids encoding the protein of the invention, a regulatory element (promoter), an intron and a poly-adenylation signal. Methods for generating cells having targeted gene modifications through homologous recombination are known in the art. For non-human embryonic stem (ES) cells, an ES cell line may be employed, or embryonic cells may be obtained freshly from a host, e.g. mouse, rat, guinea pig, etc. Such cells are grown on an appropriate fibroblast-feeder layer and are grown in the presence of leukemia inhibiting factor (LIF).

When non-human ES or embryonic cells or somatic pluripotent stem cells have been transfected, they may be used to produce transgenic animals. After transfection, the cells are plated onto a feeder layer in an appropriate medium. Cells containing the construct may be selected by employing a selective medium. After sufficient time for colonies to grow, they are picked and analyzed for the occurrence of homologous recombination or integration of the construct. Those colonies that are positive may then be used for embryo transfection and morula aggregation. Briefly, morulae are obtained from 4 to 6 week old superovulated females, the Zona Pellucida is removed and the morulae are put into small depressions of a tissue culture dish. The ES cells are trypsinized, and the modified cells are placed into the depression closely to the morulae. On the following day the aggregates are transfered into the uterine horns of pseudopregnant females. Females are then allowed to go to term. Chimeric offsprings can be readily detected by a change in coat color and are subsequently screened for the transmission of the mutation into the next generation (F1-generation). Offspring of the F1-generation are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc., for example, mouse, rat, guinea pig, sheep, cow, pig, and others. The transgenic animals may be used in functional studies, drug screening, and other applications and are useful in the study of the function and regulation of the protein of the invention in vivo.

Finally, the invention also relates to a kit comprising at least one of
(a) a nucleic acid molecule coding for the protein of the invention or a functional fragment thereof;
(b) the protein of the invention or a functional fragment or an isoform thereof;
(c) a vector comprising the nucleic acid of (a);
(d) a host cell comprising the nucleic acid of (a) or the vector of (c);
(e) a polypeptide encoded by the nucleic acid of (a);
(f) a fusion polypeptide encoded by the nucleic acid of (a);
(g) an antibody, an aptamer or another effector/modulator of the nucleic acid of (a) or the polypeptide of (b), (e), or (f) and
(h) an anti-sense oligonucleotide of the nucleic acid of (a).

The kit may be used for diagnostic or therapeutic purposes or for screening applications as described above. The kit may further contain user instructions.

The Figures show:

FIG. 1 shows the content of energy storage metabolites (ESM; triglyceride (TG) and glycogen) of Drosophila PRL-1 (GadFly Accession Number CG4993) mutants. Shown is the change of triglyceride content of HD-EP(2)20261 flies caused by integration of the P-vector into the into the annotated transcription unit (column 3) in comparison to controls containing more than 2000 fly lines of the proprietary EP collection (‘HD-control (TG)’, column 1) and wildtype controls determined in more than 80 independent assays (referred to as ‘WT-control (TG)’ column 2). Also shown is the change of glycogen content of HD-EP(2)20261 flies caused by integration of the P-vector the into the annotated transcription unit (column 5) in comparison to controls (referred to as ‘control (glycogen)’ column 4).

FIG. 2 shows the molecular organization of the mutated PRL-1 (Gadfly Accession Number CG4993) gene locus.

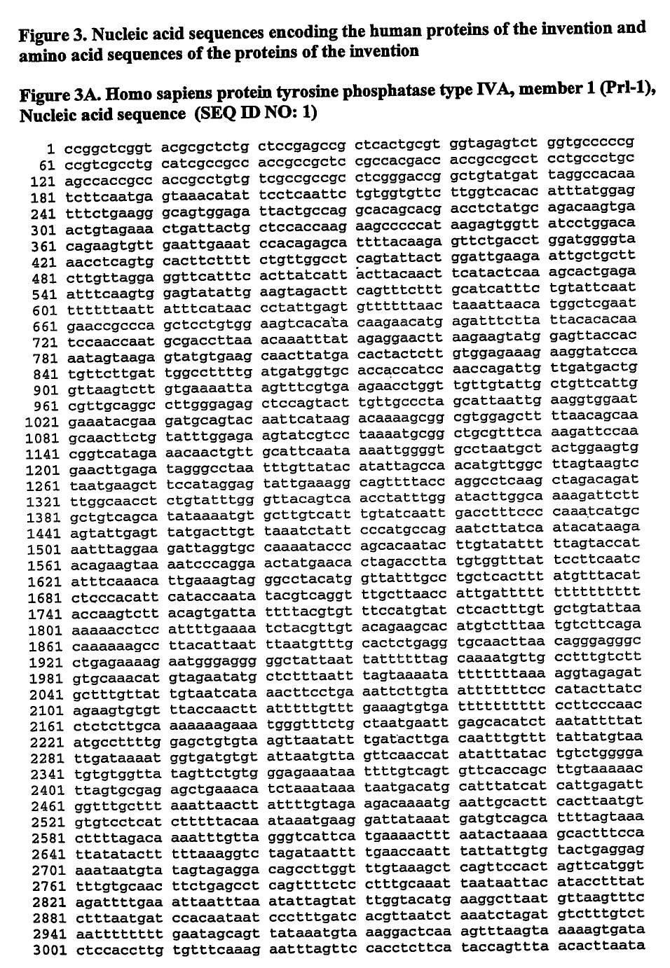

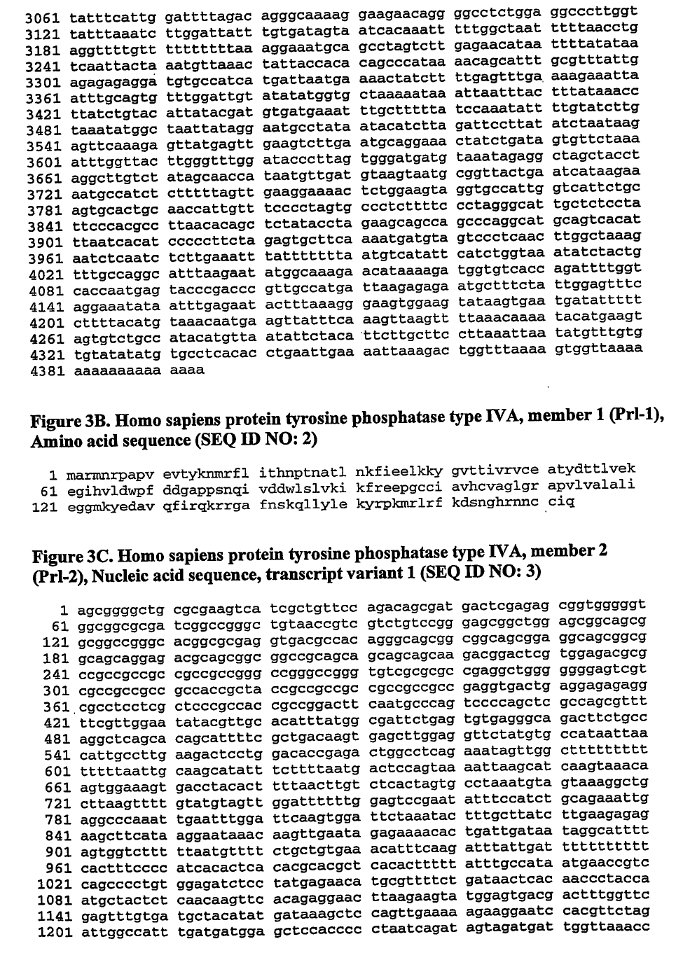

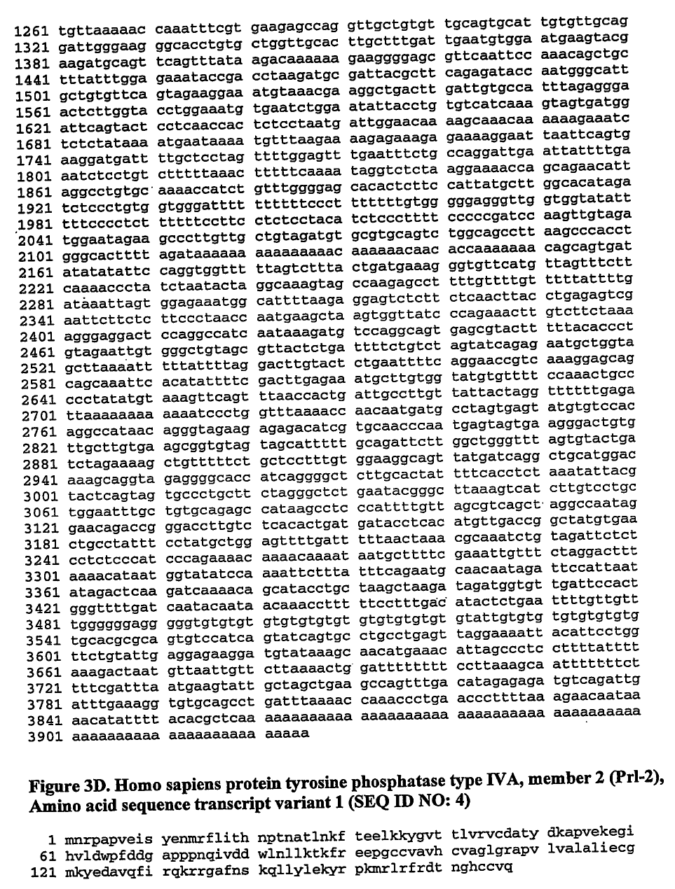

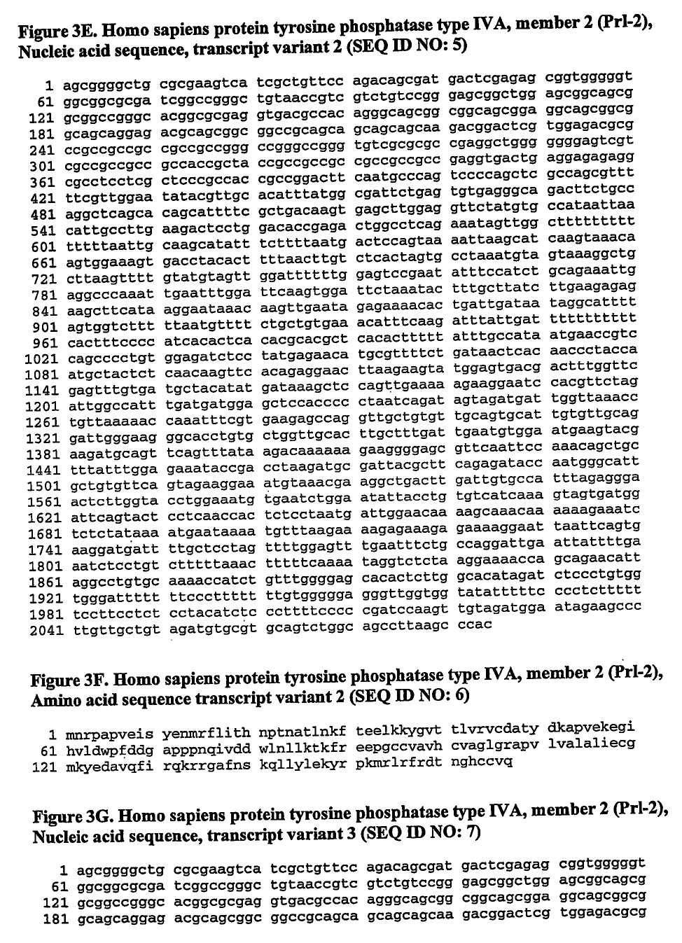

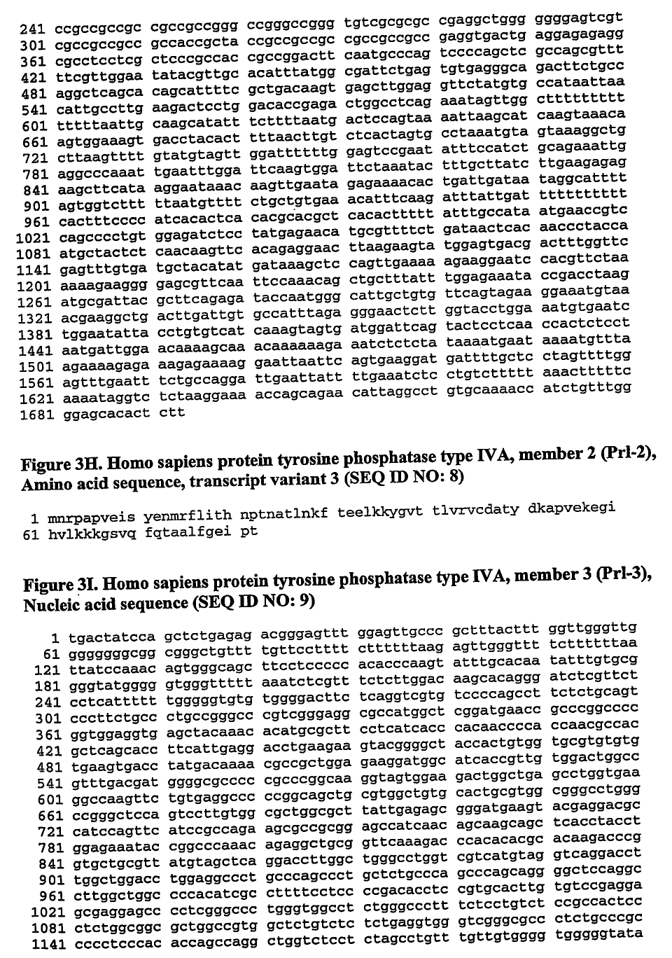

FIG. 3 shows the nucleic acid and amino acid sequences of the proteins of the invention.

FIG. 3A shows the nucleic acid sequence of human Prl-1 ( SEQ ID NO: 1).

FIG. 3B shows the amino acid sequence (one-letter code) of human Prl-1 (SEQ ID NO: 2).

FIG. 3C shows the nucleic acid sequence of human Prl-2, transcript variant 1 (SEQ ID NO: 3)

FIG. 3D shows the amino acid sequence (one-letter code) of human Prl-2, transcript variant 1 (SEQ ID NO: 4).

FIG. 3E shows the nucleic acid sequence of human Prl-2, transcript variant 2 (SEQ ID NO: 5)

FIG. 3F shows the amino acid sequence (one-letter code) of human Prl-2, transcript variant 2 (SEQ ID NO: 6).

FIG. 3G shows the nucleic acid sequence of human Prl-2, transcript variant 3 (SEQ ID NO: 7)

FIG. 3H shows the amino acid sequence (one-letter code) of human Prl-2, transcript variant 3 (SEQ ID NO: 8).

FIG. 3I shows the nucleic acid sequence of human Prl-3 ( SEQ ID NO: 9)

FIG. 3J shows the amino acid sequence (one-letter code) of human Prl-3 (SEQ ID NO: 10).

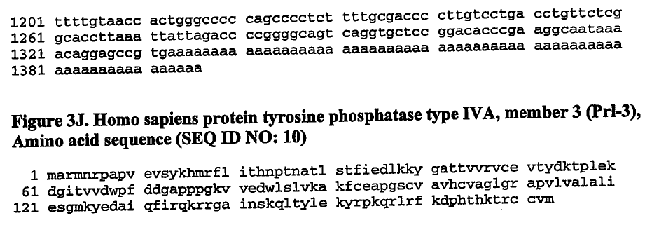

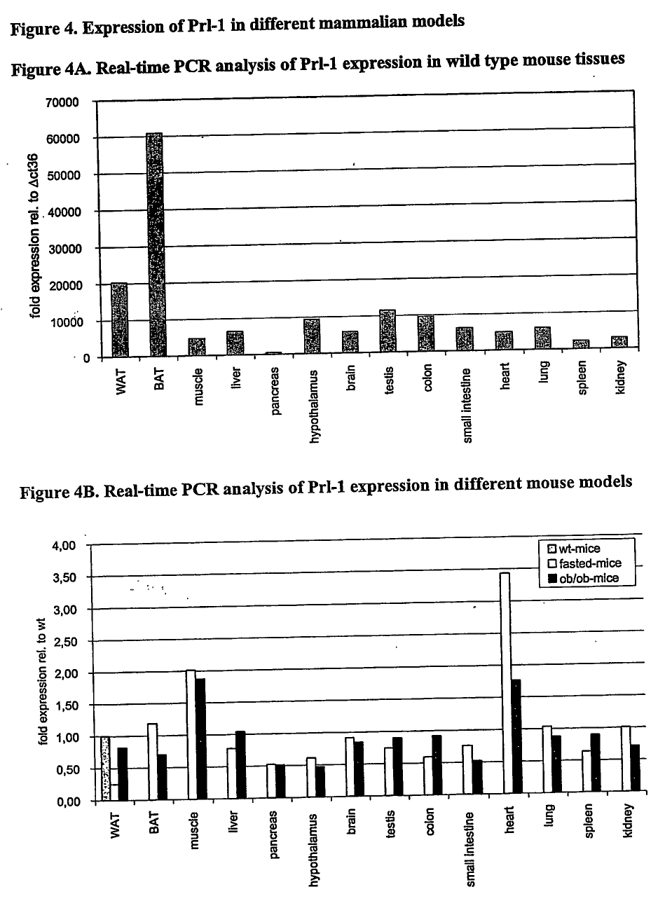

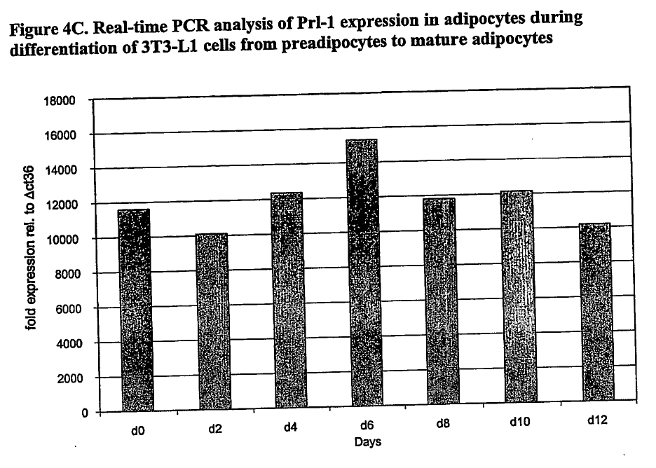

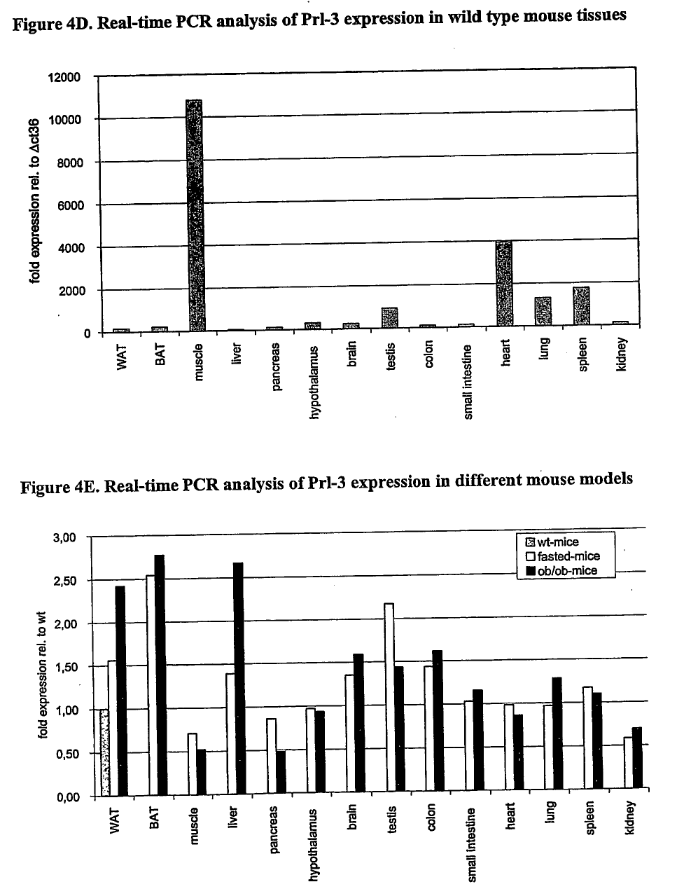

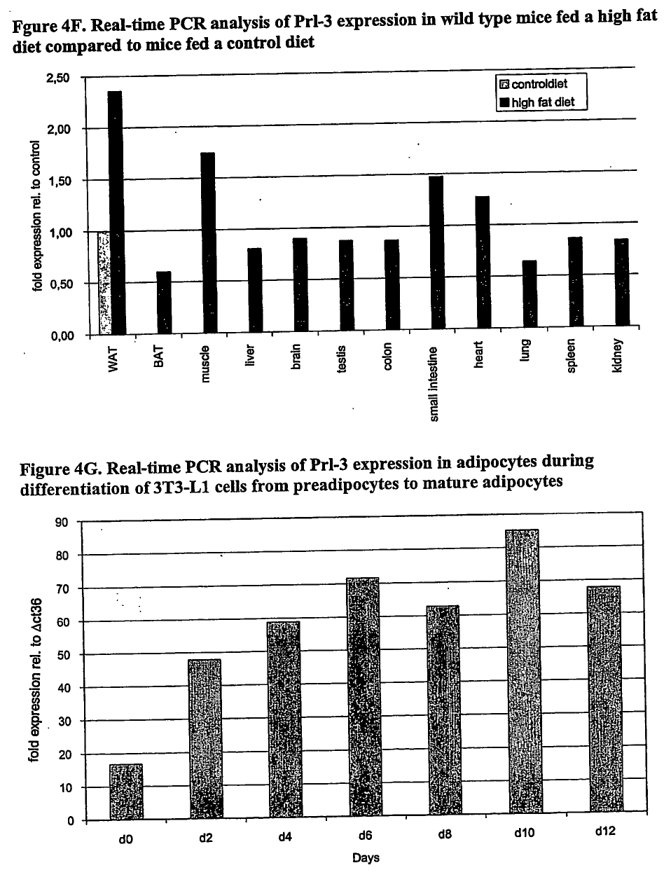

FIG. 4 shows the expression of Prl-1 in different mammalian models

FIG. 4A shows real-time PCR analysis of Prl-1 expression in wildtype mouse tissues. The relative RNA-expression is shown on the Y-axis, the tissues tested are given on the X-axis. (WAT=white adipose tissue, light grey columns; BAT=brown adipose tissue, dark grey columns)

FIG. 4B shows real-time PCR analysis of Prl-1 expression in different mouse models.

FIG. 4C shows real-time PCR analysis of Prl-1 expression in adipocytes during differentiation of 3T3-L1 cells from preadipocytes to mature adipocytes.

FIG. 4D shows real-time PCR analysis of Prl-3 expression in wild type mouse tissues.

FIG. 4E shows real-time PCR analysis of Prl-3 expression in different mouse models.

FIG. 4F shows real-time PCR analysis of Prl-3 expression in wild type mice fed a high fat diet compared to mice fed a control diet.

FIG. 4G shows real-time PCR analysis of Prl-3 expression in adipocytes during differentiation of 3T3-L1 cells from preadipocytes to mature adipocytes.

FIG. 5 shows in vitro assays for the determination of triglyceride and glycogen levels in cells overexpressing Prl-1.

FIG. 5A shows an increase in triglyceride levels in cells overexpressing Prl-1. The Y-axis shows cellular triglyceride (shown as microg triglyceride per mg protein) levels and the X-axis shows days of cell differentiation. Measurements from cells overexpressing Prl-1 are shown as dark grey columns, control cells (empty vector) are shown as light grey columns. Triglyceride levels and controls are shown for two different sets of samples.

FIG. 5B shows an increase in glycogen levels in cells overexpressing Prl-1. The Y-axis shows glycogen levels (shown as microg glycogen per mg protein) and the X-axis shows days of cell differentiation. Measurements from cells overexpressing Prl-1 are shown as dark grey columns, control cells (empty vector) are shown as light grey columns. Glycogen levels and controls are shown for two different sets of samples.

FIG. 6 shows in vitro assays for the determination of lipid synthesis and fatty acid esterification levels in Prl-1 loss of function (LOF) cells.

FIG. 6A shows a decrease in lipid synthesis levels in Prl-1 LOF cells. The Y-axis shows the amount of synthesized lipids (shown as dpm per mg protein) and the X-axis shows the controls and Prl-1 LOF cells. Measurements from insulin-stimulated samples are shown as dark grey columns, basic samples are shown as light grey columns. Lipid levels and controls are shown for three different sets of samples.

FIG. 6B shows an increase in fatty acid (FA) esterification levels in Prl-1 LOF cells. The Y-axis shows the amount of esterified fatty acids (shown as dpm per mg protein) and the X-axis shows controls and Prl-1 LOF cells. FA esterification levels are shown for three different sets of samples.

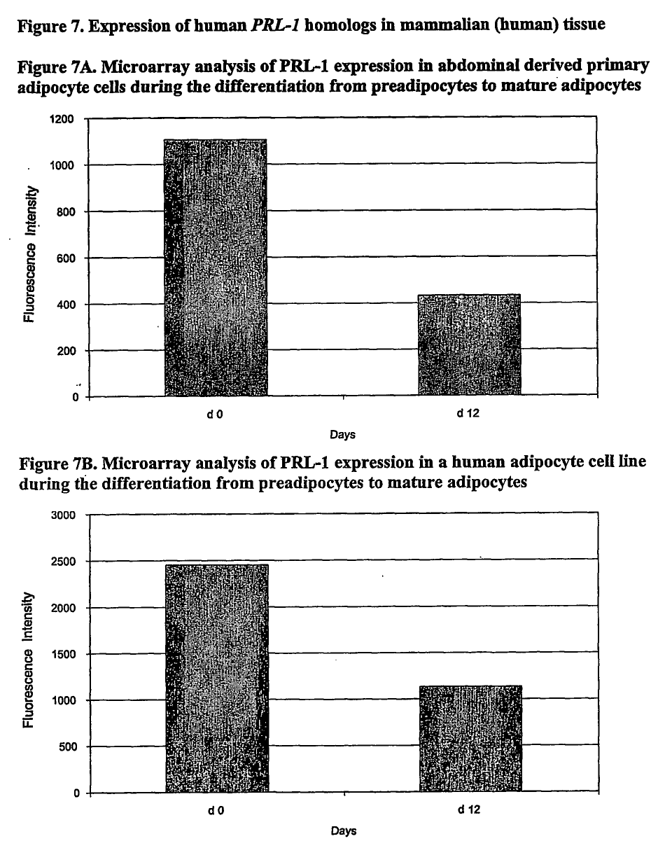

FIG. 7 shows the expression of human PRL-1 homologs in mammalian (human) issue.

FIG. 7A shows the microarray analysis of PRL-1 expression in abdominal derived primary adipocyte cells during the differentiation from preadipocytes to mature adipocytes.

FIG. 7B shows the microarray analysis of PRL-1 expression in a human adipocyte cell line during the differentiation from preadipocytes to mature adipocytes.

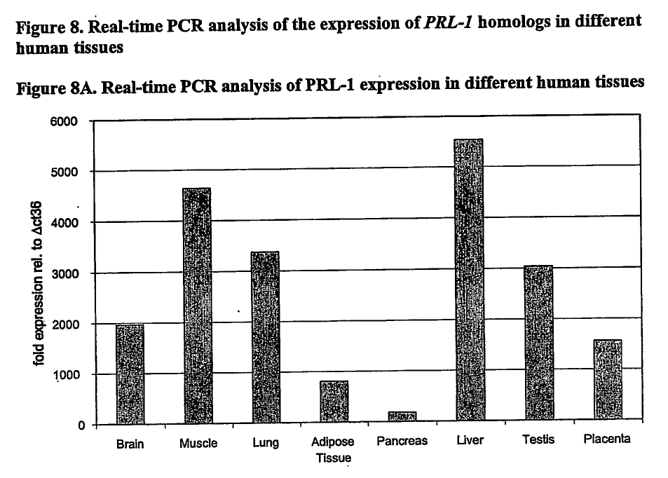

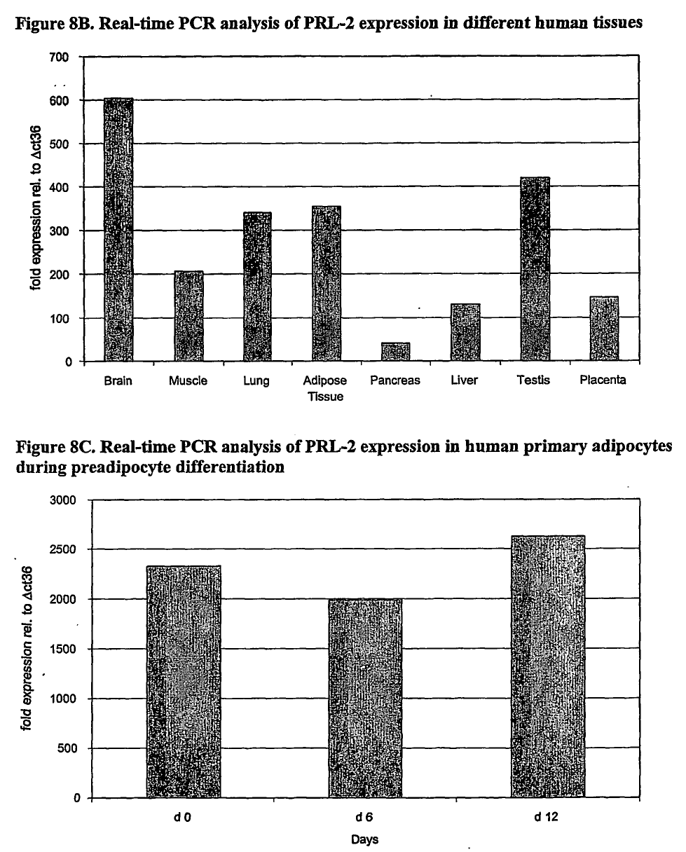

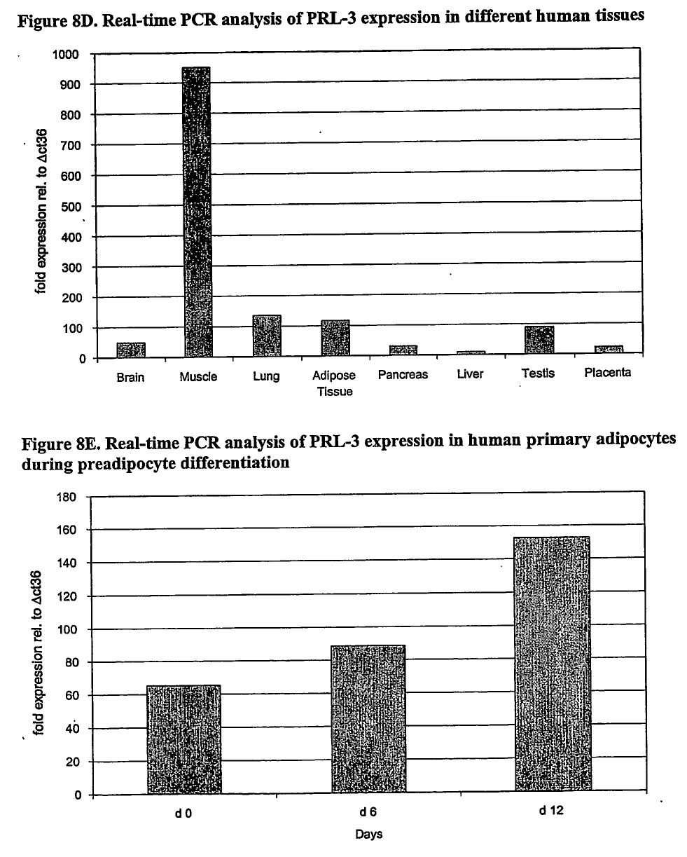

FIG. 8 shows real-time PCR analysis of the expression of PRL-1 homologs in different human tissues.

FIG. 8A shows real-time PCR analysis of PRL-1 expression in human tissues.

FIG. 8B shows real-time PCR analysis of PRL-2 expression in different human tissues.

FIG. 8C shows real-time PCR analysis of PRL-2 expression in human primary adipocytes during preadipocyte differentiation.

FIG. 8D shows real-time PCR analysis of PRL-3 expression in different human tissues.

FIG. 8E shows real-time PCR analysis of PRL-3 expression in human primary adipocytes during preadipocyte differentiation.

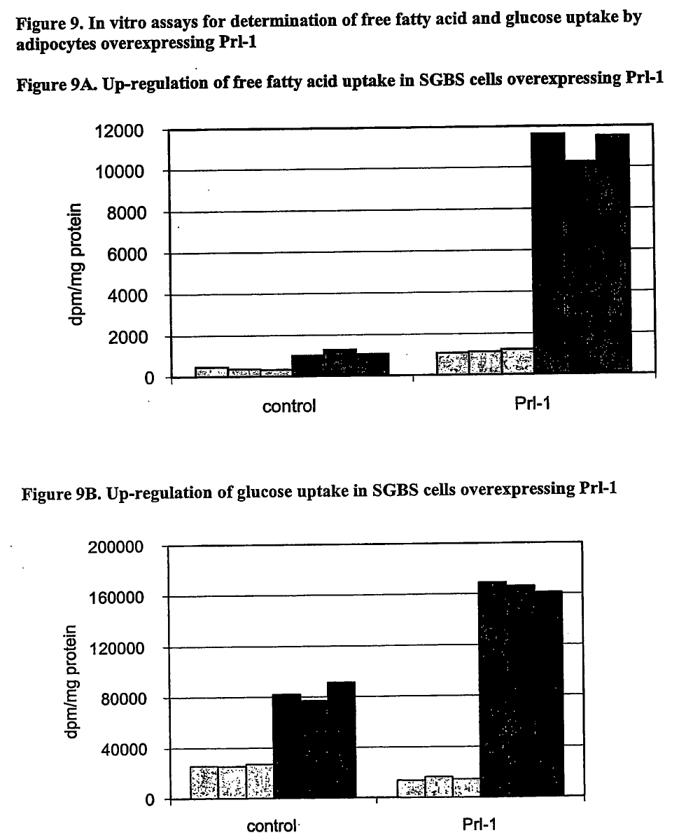

FIG. 9 shows in vitro assays for the determination of free fatty acid and glucose uptake by adipocytes overexpressing Prl-1.

FIG. 9A shows an increase in free fatty acid uptake by Prl-1 overexpressing SGBS cells. The Y-axis shows the ³H-oleic acid uptake (radioactivity in dpm/mg protein) and the X-axis the kind of analysed cells: control cells (empty vector) and Prl-1 overexpressing cells. Passive diffusion of ³H-oleic acid through the plasma membrane is shown as light grey columns, active transport as dark grey columns. The ³H-oleic acid uptake is shown for three different sets of samples.

FIG. 9B shows an increase in glucoseuptake by Prl-1 overexpressing SGBS cells. The Y-axis shows the 2-deoxy-³H-D-glucose uptake (radioactivity in dpm/mg protein) and the X-axis the kind of analysed cells: control cells (empty vector) and PRL-1 overexpressing cells. Basal 2-deoxy-³H-D-glucose uptake is shown as light grey columns, insulin stimulated uptake as dark grey columns. The 2-deoxy-³H-D-glucose uptake is shown for three different sets of samples.

### THE EXAMPLES ILLUSTRATE THE INVENTION

### Example 1

### Measurement of Energy Storage Metabolites (ESM) Contents in Drosophila

Mutant flies are obtained from a fly mutation stock collection. The flies are grown under standard conditions known to those skilled in the art. In the course of the experiment, additional feedings with bakers yeast (Saccharomyces cerevisiae) are provided for the EP-line HD-EP20261. The average change of triglyceride and glycogen (herein referred to as energy storage metabolites, ESM) content of Drosophila containing the EP-vector as homozygous viable integration was investigated in comparison to control flies, respectively (see FIG. 1). For determination of ESM content, flies were incubated for 5 min at 90° C. in an aqueous buffer using a waterbath, followed by hot extraction. After another 5 min incubation at 90° C. and mild centrifugation, the triglyceride content of the flies extract was determined using Sigma Triglyceride (INT 336-10 or -20) assay by measuring changes in the optical density according to the manufacturer's protocol, and the glycogen content of the flies extract was determined using Roche (Starch UV-method Cat. No. 0207748) assay by measuring changes in the optical density according to the manufacturer's protocol. As a reference the protein content of the same extract was measured using BIO-RAD DC Protein Assay according to the manufacturer's protocol. These experiments and assays were repeated several times.

The average triglyceride level (μg triglyceride/μg protein) of 2108 fly lines of the proprietary EP-collection (referred to as ‘HD-control (TG)’) is shown as 100% in the first column in FIG. 1. The average triglyceride level (μg triglyceride/μg protein) of Drosophila wildtype strain Oregon R flies determined in 84 independent assays (referred to as ‘WT-control (TG)’) is shown as 102% in the second column in FIG. 1. The average glycogen level (μg glycogen/μg protein) of an internal assay control consisting of two different wildtype strains and an inconspicuous EP-line of the HD stock collection (referred to as ‘control (glycogen)’) is shown as 100% in the fourth column in FIG. 1. Standard deviations of the measurements are shown as thin bars. HD-20261 homozygous flies (column 3 in FIG. 1, ‘HD-20261 (TG)’) show constantly a higher triglyceride content (μg triglyceride/μg protein) than the controls. HD-20261 homozygous flies (column 5 in FIG. 1, ‘HD-20261 (glycogen)’) also show a lower glycogen content (μg glycogen/μg protein) than the controls. Therefore, the loss of gene activity is responsible for changes in the metabolism of the energy storage metabolites.

### Example 2

### Identification of a Drosophila Gene Responsible for Changes in Triglyceride and Glycogen Levels

Genomic DNA sequences were isolated that are localized directly adjacent to the EP vector (herein HD-EP(2)20261) integration. Using those isolated genomic sequences public databases like Berkeley Drosophila Genome Project (GadFly) were screened thereby confirming the homozygous viable integration site of the HD-EP(2)20261 vector at base pair 9 of the transcript variant CG4993-RB of the gene PRL-1 in sense orientation. FIG. 2 shows the molecular organization of this gene locus. The chromosomal localization site of integration of the vector of HD-EP(2)20261 is at gene locus 2L, 35E2 (according to Flybase) or 2L, 35F1 (according toGadfly release 3). In FIG. 2, genomic DNA sequence is represented by the assembly as a black arrow in middle of the figure that includes the integration site of HD-EP(2)20261. Ticks represent the length in base pairs of the genomic DNA (1000 base pairs per tick). Dark grey bars in the lower half of the figure, linked by dark grey lines represent cDNAs of the predicted genes (as predicted by the Berkeley Drosophila Genome Project, GadFly release 3). Predicted exons of the Drosophila cDNA of the gene PRL-1 (GadFly Accession Number CG4993) are shown as dark grey bars and predicted introns as slim grey lines in the lower half of the figure and are labeled. The integration site of HD-EP20261 is indicated with a black triangle within the first exon of the predicted cDNA of the PRL-1 gene. The corresponding expressed sequence tags (ESTs) are shown as light grey bars below the two cDNA transcript variants. Therefore, expression of the cDNA encoding PRL-1 could be affected by integration of the vector of line HD-EP(2)20261, leading to a change in the amount of energy storage triglycerides.

Table 1 is summarizing the data of our molecular analysis of the Drosophila protein identified in this invention as being involved in the regulation of the metabolism.

TABLE 1
Molecular analysis of Drosophila PRL-1

| | |
|---|---|
| Analysis | Prl-1 |
| Protein | prenylated protein tyrosine phosphatase |
| | (Flybase) |
| Protein | Dual specificity protein phosphatase, |
| domains | Tyrosine specific protein phosphatase and |
| | dual specificity protein phosphatase |
| | family, (Phosphotyrosine protein) |
| | phosphatases II (Flybase) |
| InterPro | Tyrosine specific protein phosphatase and |
| analysis | dual specificity protein phosphatase |
| | (IPR000387) |
| Functional | not described (Flybase) |
| data | |
| Locus | 2L, 35E2 (Flybase); 2L, 35F1 (Gadfly |
| | release 3) |
| ESTs | many including RE55984 (Gadfly release 3) |
| CDNA | AA140816, AA392622,, AA538688, AA540959, |
| | AA696442, AA803899, AA821150, AA821153, |
| | AA979376, AA979397, AA979413, AA990900, |
| | AF063902, AF390535, AI063355, AY071505, |
| | BI369716 (Flybase) |
| Genomic DNA | AE003415, AE003650 (Flybase) |
| NCBI locus | 34952, Dm Prl-1, 35E2. |
| ID | Aliases: PRL, CG4993, CT16026, |
| | BG: DS07473.3 |
| | RefSeq: NM_135936 |
| | Nucleotide: AE003415, AE003650, AQ025903, |
| | AQ025971, AQ073277, AQ074056, AA140816, |
| | AA392622, AA538688, AA540959, AA696442, |
| | AA803899, AA821150, AA821153, AA979376, |
| | AA979397, AA979413, AA990900, AF063902, |
| | AF390535, AY071505 |
| | Protein: NP_609780, AAF44989, AAF53506, |
| | AAC16552, AAL26988, AAL49127 |
| Mutations | 5 recorded alleles: 4 classical mutants |
| and mutants | and 1 wild-type (Flybase) |

### Example 3

### Identification of the Human PRL-1 Homologous Proteins

PRL-1 homologous proteins and nucleic acid molecules coding therefore are obtainable from insect or vertebrate species, e.g. mammals or birds. Particularly preferred are nucleic acids comprising Drosophila PRL-1 or human Prl-1, Prl-2, and Prl-3 and homologs. Sequences homologous to Drosophila PRL-1 were identified using the publicly available program BLASTP 2.2.3 of the non-redundant protein data base of the National Center for Biotechnology Information (NCBI) (see, Altschul et al., 1997, Nucleic Acids Res. 25:3389-3402). Table 2 shows the best human homologs of the Drosophila PRL-1 gene.

TABLE 2
Human homologous proteins to Drosophila PRL-1 protein

| PTP4A1 | NCBI human locus identification (ID): 7803, |
|---|---|
| | Hs PTP4A1, protein tyrosine phosphatase type |
| | IVA, member 1, 6q12 |

Aliases: HH72, PRL1, Prl-1, PTP4A2, PTPCAAX1,
PTP(CAAX1)
OMIM: 601585
RefSeq[R]: NM_003463
Nucleotide: AF051160, AJ420505, BC023975,
BI222469, U48296, U69701
Protein: NP_003454, AAC39836, AAH23975,
AAB40597, AAB09080

| PTP4A2 | NCBI human locus identification (ID): 8073, |
|---|---|
| | Hs PTP4A2, protein tyrosine phosphatase type |
| | IVA, member 2, 1p35 |

Aliases: HH13, OV-1, PRL2, HH7-2, PRL-2,
PTP4A, HU-PP-1, PTPCAAX2, ptp-IV1a, ptp-IV1b
OMIM: 601584
RefSeq[R]: NM_003479, NM_080391,
NM_080392
Nucleotide: L48722, L48723, L48937, AF208850,
BI552091, L39000, U14603, U48297
Protein: NP_003470, NP_536316, NP_536317,
AAB42169, AAB42170, AAB39331, AAF64264,
AAB59575, AAA90979, AAB40598

| PTP4A3 | NCBI human locus identification (ID): 11156, |
|---|---|
| | Hs PTP4A3, protein tyrosine phosphatase type |
| | IVA, member 3 |

Aliases: PRL3, PRL-3, PRL-R
OMIM: 606449
RefSeq[R]: NM_007079, NM_032611
Nucleotide: AF041434, AI742376, BC003105,
BE778111, U87168
Protein: NP_009010, NP_116000, AAC29314,
AAH03105, AAB47560

The mouse homologous cDNAs encoding the polypeptides of the invention were identified as GenBank Accession Numbers NM_{—}011200, XM_{—}123656, XM_{—}135289, NM_{—}008974, NM_{—}008975.

### Example 4

### Expression of PRL-1 Homologous mRNA in Mammalian Tissues

### Example 4A

### Prl-1 mRNA Expression in Mammalian (Mouse) Tissues (FIG. 4)

To analyse the expression of the PRL-1 homologous mRNA disclosed in this invention in mammalian tissues, several mouse strains (preferably mice strains C57BI/6J, C57BI/6 ob/ob and C57BI/KS db/db which are standard model systems in obesity and diabetes research) were purchased from Harlan Winkelmann (33178 Borchen, Germany) and maintained under constant temperature (preferably 22° C.), 40 per cent humidity and a light/dark cycle of preferably 14/10 hours. The mice were fed a standard chow (for example, from ssniff Spezialitäten GmbH, order number ssniff M-Z V1126-000). For the fasting experiment (“fasted wild type mice”), wild type mice were starved for 48 h without food, but only water supplied ad libitum. (see, for example, Schnetzler B. et al., (1993) J Clin Invest 92: 272-280, Mizuno T. M. et al., (1996) Proc Natl Acad Sci USA 93: 3434-3438). In a further experiment wild-type (wt) mice were fed a control diet (preferably Altromin C1057 mod control, 4.5% crude fat) or high fat diet (preferably Altromin C1057mod. high fat, 23.5% crude fat). Animals were sacrificed at an age of 6 to 8 weeks. The animal tissues were isolated according to standard procedures known to those skilled in the art, snap frozen in liquid nitrogen and stored at −80° C. until needed.

For analyzing the role of the proteins disclosed in this invention in the in vitro differentiation of different mammalian cell culture cells for the conversion of preadipocytes to adipocytes, mammalian fibroblast (3T3-L1) cells (e.g., Green H. and Kehinde O., (1974) Cell 1: 113-116) were obtained from the American Tissue Culture Collection (ATCC, Hanassas, Va., USA; ATCC-CL 173). 3T3-L1 cells were maintained as fibroblasts and differentiated into adipocytes as described in the prior art (e.g., Qiu. Z. et al., (2001) J. Biol. Chem. 276: 11988-11995; Slieker L. J. et al., (1998) BBRC 251: 225-229). In brief, cells were plated in DMEM/10% FCS (Invitrogen, Karisruhe, Germany) at 50,000 cells/well in duplicates in 6-well plastic dishes and cultured in a humidified atmosphere of 5% CO₂ at 37° C. At confluence (defined as day 0: d0) cells were transferred to serum-free (SF) medium, containing DMEM/HamF12 (3:1; Invitrogen), fetuin (300 μg/ml; Sigma, Munich, Germany), transferrin (2 μg/ml; Sigma), pantothenate (17 μM; Sigma), biotin (1 μM; Sigma), and EGF (0.8 nM; Hoffmann-La Roche, Basel, Switzerland). Differentiation was induced by adding dexamethasone (DEX; 1 μM; Sigma), 3-methyl-isobutyl-1-methylxanthine (MIX; 0.5 mM; Sigma), and bovine insulin (5 μg/ml; Invitrogen). Four days after confluence (d4), cells were kept in SF medium, containing bovine insulin (5 μg/ml) until differentiation was completed. At various time points of the differentiation procedure, beginning with day 0 (day of confluence) and day 2 (hormone addition; for example, dexamethasone and 3-isobutyl-1-methylxanthine), up to 10 days of differentiation, suitable aliquots of cells were taken every two days.

RNA was isolated from mouse tissues or cell culture cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art. Total RNA was reverse transcribed (preferably using Superscript II RNaseH-Reverse Transcriptase, from Invitrogen, Karlsruhe, Germany) and subjected to Taqman analysis preferably using the Taqman 2×PCR Master Mix (from Applied Biosystems, Weiterstadt, Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

The following primer/probe pairs were used for the TaqMan analysis (GenBank Accession Number U84411 for the mouse Prl-1 sequence):

Mouse Prl-1 forward primer (Seq ID NO:11):
5′- GCT GTA TTG CTG TCC ATT GTG TC -3′;
mouse Prl-1 reverse primer (Seq ID NO:12):
5- TCC ACC TTC AAT TAA TGC TAG GG -3′;
mouse Prl-1 Taqman probe (Seq ID NO:13):
(5/6-FAM)- CAG GCC TTG GCA GAG CTC CGG -
(5/6-TAMRA).

The following primer/probe pairs were used for the TaqMan analysis (GenBank Accession Number NM_{—}008975 for the mouse Prl-3 sequence):

Mouse Prl-3 forward primer (Seq ID NO:14):
5′- AGC TAC CGG CAC ATG CG-3′;
mouse Prl-3 reverse primer (Seq ID NO:15):
5′- ACG TGC TGA GGG TGG CA-3′;
mouse Prl-3 Taqman probe (Seq ID NO:16):
(5/6-FAM)- TCC TCA TCA CCC ACA ACC CCA GC-
(5/6-TAMRA).

In FIG. 4 the relative RNA-expression is shown on the Y-axis. In FIGS. 4A-B and 4D-F, the tissues tested are given on the X-axis. “WAT” refers to white adipose tissue, “BAT” refers to brown adipose tissue. In FIGS. 4C and 4G, the X-axis represents the time axis. “d0” refers to day 0 (start of the experiment), “d2”-“d12” refers to day 2-day 12 of adipocyte differentiation.

The function of the proteins of the invention in metabolism was further validated by analyzing the expression of the transcripts in different tissues and by analyzing the role in adipocyte differentiation.

Mouse models of insulin resistance and/or diabetes were used, such as mice carrying gene knockouts in the leptin pathway (for example, ob/ob (leptin) or db/db (leptin receptor/ligand) mice) to study the expression of the proteins of the invention. Such mice develop typical symptoms of diabetes, show hepatic lipid accumulation and frequently have increased plasma lipid levels (see Bruning J. C. et al, (1998) Mol. Cell. 2: 559-569).

Expression of the mRNAs encoding the proteins of the invention was also examined in susceptible wild type mice (for example, C57BI/6) that show symptoms of diabetes, lipid accumulation, and high plasma lipid levels, if fed a high fat diet.

Expression profiling studies confirm the particular relevance of PRL-1 homologous proteins as regulators of energy metabolism in mammals.

Taqman analysis revealed that Prl-1 is expressed in several mammalian tissues, showing highest level of expression in brown adipose tissue (BAT) compared to other tissue types in wild type mouse, and high expression levels in further tissues, e.g. white adipose tissue (WAT), muscle, liver, hypothalamus, brain, testis, colon, small intestine, heart, lung, spleen, and kidney. Furthermore Prl-1 is expressed on lower but still robust levels in the pancreas of wild type mice as depicted in FIG. 4A. This high expression of Prl-1 in BAT confirms an essential role of Prl-1 in adipogenesis.

We found, for example, that the expression of Prl-1 is down-regulated in the WAT, and up-regulated in heart and muscle of fasted mice compared to wild type mice. Furthermore the expression of Prl-1 is down-regulated in the hypothalamus of genetically induced obese mice (ob/ob) compared to wild type mice (see FIG. 4B). We show in this invention (see FIG. 4C) that the Prl-1 mRNA is expressed during the differentiation into mature adipocyctes. Therefore, the Prl-1 protein might play a role in adipogenesis.

The expression of Prl-1 in metabolic active tissues of wild type mice, as well as the regulation of Prl-1 in different animal models used to study metabolic disorders, suggests that this gene plays a central role in energy homeostasis. This hypothesis is supported by the expression during the differentiation from preadipocytes to mature adipocytes.

Taqman analysis revealed that Prl-3 is expressed in several mammalian tissues, showing highest level of expression in muscle and higher levels in further tissues, e.g. heart, spleen, lung, and testis of wild type mice. Furthermore Prl-3 is expressed on lower but still robust levels in WAT, BAT, liver, pancreas, hypothalamus, brain, colon, small intestine, and kidney of wild type mice as depicted in FIG. 4D. We found, for example, that the expression of Prl-3 is up-regulated in the WAT, BAT, and liver, and slightly down-regulated in the muscle and pancreas of genetically induced obese mice (ob/ob) compared to wild type mice. Furthermore Prl-3 is up-regulated in the testis and BAT of fasted mice compared to wild type mice (see FIG. 4E). In wild type mice fed a high fat diet, the expression of Prl-3 is up-regulated in WAT as depicted in FIG. 4F. We show in this invention (see FIG. 4G) that the Prl-3 mRNA is expressed and up-regulated during the differentiation into mature adipocyctes. Therefore, the Prl-3 protein might play an essential role in adipogenesis.

The expression of Prl-3 is regulated in metabolic active tissues (e.g. WAT and BAT) of different animal models used to study metabolic disorders, together with the regulated expression during the differentiation from preadipocytes to mature adipocytes, suggests that this gene plays a central role in energy homeostasis.

### Example 4B

### PRL-1 mRNA Expression In Mammalian (Human) Tissues (FIG. 8)

Human primary adipocytes were differentiated into mature adipocytes as described by (Hauner et al., (1989) J Clin Invest 84: 1663-1670). Briefly, cells were grown in DMEM/Nutrient Mix F12, 1% PenStrep, 17 μM biotin, 33 μM pantothenate, 10% none heat inactivated fetal calf serum. On day 0 of differentiation, the medium was changed to DMEM/Nutrient Mix F12, 1% Pen/Strep, 17 μM biotin, 33 μM pantothenate, 0.01 mg/ml transferrin, hydrocortisone, 20 nM human insulin, 0.2 nM T3, 25 nM dexamethasone, 250 μM IBMX, 3 μM rosiglitazone. On day 4 of differentiation, the medium was changed to DMEM/Nutrient Mix F12 1% Pen/Strep, 17 μM biotin, 33 μM pantothenate, 0.01 mg/ml transferrin, 100 nM hydrocortisone, 20 nM human insulin, 0.2 nM T3. At various time points of the differentiation procedure, beginning with day 0 (day of confluence) and day 4 (hormone addition), up to 14 days of differentiation, suitable aliquots of cells were taken. RNA was isolated from human cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art.

RNAs isolated from different human tissues were obtained from Invitrogen Corp., Karlsruhe, Germany, Stratagene Amsterdam, The Netherlands, or BD Biosciences Clontech, Palo Alto, Calif., USA. (i) total RNA from human normal brain (Invitrogen Corp. Order Number D6030-01); (ii) total RNA from human adult skeletal muscle (Stratagene Order Number 735030); (iii) total RNA from human adult lung (Stratagene Order Number 735020); (iv) total RNA from human normal adipose tissue (Invitrogen Corp. Order Number D6005-01); (v) total RNA from human normal pancreas (Invitrogen Corp. Order Number D6101-01);.(vi) total RNA from human adult liver (Stratagene Order Number 735018); (vii) total RNA from human adult testis (BD Biosciences Clontech Order Number 64101-1); (viii) total RNA from human adult placenta (Stratagene Order Number 735026). The RNA was treated with DNase according to the instructions of the manufacturers (for example, from Qiagen, Germany) and as known to those skilled in the art.

Total RNA was reverse transcribed (preferrably using Superscript II RNaseH-Reverse Transcriptase, from Invitrogen, Karlsruhe, Germany) and subjected to Taqman analysis preferrably using the ,Taqman 2× PCR Master Mix’(from Applied Biosystems, Weiterstadt, Germany). The Taqman 2×PCR Master Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (all obtained from Applied Biosystems, Weiterstadt, Germany).

Taqman analysis was performed preferrably using the following primer/probe pairs:

For the amplification of human PRL-1:

human PRL-1 forward primer (SEQ ID NO: 17):
5′- TCG TGA AGA ACC TGG TTG TTG TA -3′;
human PRL-1 reverse primer (SEQ ID NO: 18):
5′- TTA ATG CTA GGG CAA CAA GTA CTG G -3′;
human PRL-1 Taqman probe (SEQ ID NO: 19):
(5/6-FAM)- TGC TGT TCA TTG CGT TGC AGG CC-
(5/6-TAMRA).

For the amplification of human PRL-2:

human PRL-2 forward primer (SEQ ID NO: 20):
5′- GGA GTG ACG ACT TTG GTT CGA -3′;
human PRL-2 reverse primer (SEQ ID NO: 21):
5′- GCC AAT CTA GAA CGT GGA TTC CT -3′;
human PRL-2 Taqman probe (SEQ ID NO: 22):
(5/6-FAM)- TTG TGA TGC TAC ATA TGA TAA AGC TCC AGT
TGA AAA AG -(5/6-TAMRA).

For the amplification of human PRL-3:

human PRL-3 forward primer (SEQ ID NO: 23):
5′- AAG TAC GAG GAC GCC ATC CA -3′;
human PRL-3 reverse primer (SEQ ID NO: 24):
5′- CTG CTT GCT GTT GAT GGC TC -3′;
human PRL-3 Taqman probe (SEQ ID NO: 25):
(5/6-FAM)- TTC ATC CGC CAG AAG CGC CG -
(516-TAMRA).

As shown in FIG. 8A, real time PCR (Taqman) analysis of the expression of PRL-1 in human tissues revealed that PRL-1 is expressed in all tissues analysed with highest levels of expression in liver and muscle, and high expression levels in further tissues, e.g. lung, testis, brain and placenta. Furthermore PRL-1 is expressed on lower but still robust levels in adipose tissue and pancreas.

The high expression of PRL-1 in metabolic active tissues (liver and muscle) suggests that this gene plays a role in energy homeostasis.

As shown in FIG. 8B, real time PCR (Taqman) analysis of the expression of PRL-2 in human tissues revealed that PRL-2 is expressed in all tissues analysed with highest levels of expression in brain and high expression levels in further tissues, e.g. testis, adipose tissue, lung and muscle. Furthermore PRL-2 is expressed on lower but still robust levels in the placenta, liver and pancreas. As shown in FIG. 8C, PRL-2 is expressed during human adipocyte differentiation.

The high expression of PRL-2 in the adipose tissue tissues, as well as the expression of PRL-2 during the differentiation from preadipocytes to mature adipocytes, suggests that this gene plays a role in energy homeostasis.

As shown in FIG. 8D, real time PCR (Taqman) analysis of the expression of PRL-3 in human tissues revealed that the protein is predominantly expressed in muscle. Furthermore PRL-3 is expressed on lower but still robust levels in the lung, adipose tissue, testis, brain, placenta, liver and pancreas. As shown in FIG. 8E, PRL-3 is up-regulated during human adipocyte differentiation.

The high expression of PRL-3 in a metabolic active tissues (muscle), as well as the up-regulation of PRL-3 during the differentiation from preadipocytes to mature adipocytes, suggests that this gene plays a role in energy homeostasis.

### Example 5

### Assays for the Determination of Metabolites

### Example 5A

### Assays for the Determination of Triglyceride and Glycogen Storage (FIG. 5)

Retroviral Infection of Preadipocytes

Packaging cells were transfected with retroviral plasmids pLPCX carrying mouse Prl-1 transgene and a selection marker using calcium phosphate procedure. Control cells were infected with pLPCX carrying no transgene. Briefly, exponentially growing packaging cells were seeded at a density of 350,000 cells per 6-well in 2 ml DMEM+10% FCS one day before transfection. 10 min before transfection chloroquine was added directly to the overlying medium (25 μM end concentration). A 250 μl transfection mix consisting of 5 μg plasmid-DNA (candidate:helper-virus in a 1:1 ratio) and 250 mM CaCl₂ was prepared in a 15 ml plastic tube. The same volume of 2×HBS (280 μM NaCl, 50 μM HEPES, 1.5 mM Na₂HPO₄, pH 7.06) was added and air bubbles were injected into the mixture for 15 sec. The transfection mix was added drop wise to the packaging cells, distributed and the cells were incubated at 37° C., 5% CO₂ for 6 hours. The cells were washed with PBS and the medium was exchanged with 2 ml DMEM+10% CS per 6-well. One day after transfection the cells were washed again and incubated for 2 days of virus collection in 1 ml DMEM+10% CS per 6-well at 32° C., 5% CO₂. The supernatant was then filtered through a 0.45 μm cellulose acetate filter and polybrene (end concentration 8 μg/ml) was added. Mammalian fibroblast (3T3-L1) cells in a sub-confluent state were overlaid with the prepared virus containing medium. The infected cells were selected for 1 week with 2 μg/ml puromycin. Following selection the cells were checked for transgene expression by western blot and immunofluorescence. Over expressing cells were seeded for differentiation.

3T3-LI cells were maintained as fibroblasts and differentiated into adipocytes as described in the prior art and supra. For analysing the role of the proteins disclosed in this invention in the in vitro assays for the determination of triglyceride storage, synthesis and transport were performed.

Preparation of Cell Lysates for Analysis of Metabolites

Starting at confluence (D0), cell media was changed every 48 hours. Cells and media were harvested 8 hours prior to media change as follows. Media was collected, and cells were washed twice in PBS prior to lyses in 600 μl HB-buffer (0.5% polyoxyethylene 10 tridecylethane, 1 mM EDTA, 0.01M NaH₂PO₄, pH 7.4). After inactivation at 70° C. for 5 minutes, cell lysates were prepared on Bio 101 systems lysing matrix B (0.1 mm silica beads; Q-Biogene, Carlsbad, USA) by agitation for 2×45 seconds at a speed of 4.5 (Fastprep FP120, Bio 101 Thermosavant, Holbrock, USA). Supernatants of lysed cells were collected after centrifugation at 3000 rpm for 2 minutes, and stored in aliquots for later analysis at −80° C.

Changes in cellular triglyceride levels during adipogenesis (FIG. 5A) Cell lysates and media were simultaneously analysed in 96-well plates for total protein and triglyceride content using the Bio-Rad DC Protein assay reagent (Bio-Rad, Munich, Germany) according to the manufacturer's instructions and a modified enzymatic triglyceride kit (GPO-Trinder; Sigma) briefly final volumes of reagents were adjusted to the 96-well format as follows: 10 μl sample was incubated with 200 μl reagent A for 5 minutes at 37° C. After determination of glycerol (initial absorbance at 540 nm), 50 μl reagent B was added followed by another incubation for 5 minutes at 37° C. (final absorbance at 540 nm). Glycerol and triglyceride concentrations were calculated using a glycerol standard set (Sigma) for the standard curve included in each assay.

As shown in FIG. 5A, we found that in Prl-1 overexpressing cells cellular triglyceride levels were increased on day 12 of differentiation when compared to control cells which were transduced with empty vector. An increase in triglyceride levels of about 20% in these experiments is significant. When we overexpress known regulators of adipogenesis such as for example PPAR gamma-1 in 3T3-L1 cells, we repeatedly observe increases in triglyceride content between 20 and 30% as compared to control cells.

Furthermore, PRL-1 overexpression in human SGBS cells (Wabitsch et al., 2001) led to an even more pronounced phenotype (increase) on cellular triglyceride levels on day 12 of differentiation when compared to control cells, which were transduced with empty vector.

Changes in cellular glycogen levels during adipogenesis (FIG. 5B) Cell lysates and media were simultaneously analysed in triplicates in 96-well plates for total protein and glycogen content using the Bio-Rad DC Protein assay reagent (Bio-Rad, Munich, Germany) according to the manufacturer's instructions and an enzymatic starch kit from Hoffmann-La Roche (Basel, Switzerland). 10 μl samples were incubated with 20 μl amyloglucosidase solution for 15 minutes at 60° C. to digest glycogen to glucose. The glucose is further metabolised with 100 μl distilled water and 100 μl of enzyme cofactor buffer and 12 μl of enzyme buffer (hexokinase and glucose phosphate dehydrogenase). Background glucose levels are determined by subtracting values from a duplicate plate without the amyloglucosidase. Final absorbance is determined at 340 nm. HB-buffer as blank, and a standard curve of glycogen (Hoffmann-La Roche) were included in each assay. Glycogen content in the samples was calculated using a standard curve.

As shown in FIG. 5B, we found that in Prl-1 overexpressing cells cellular glycogen levels were increased throughout adipogenesis. Glycogen levels in cells are more variable than triglyceride levels because the turnover of glycogen is higher. Glucose is taken up by the cells rapidly and stored in the form of glycogen. This energy storage is then used as a first quick response to the metabolic demands of the cell. On day 12 of differentiation intracellular glycogen levels are increased by more than 100% indicating that the Prl-1 affects central metabolic pathways in the cell.

### Example 5B

### Assays for the Determination of Free Fatty Acid and Glucose Uptake

Lentiviral Infection of Preadipocytes

Packaging cells were transfected with retroviral plasmids pLenti6/V5-DEST carrying mouse Prl-1 transgene (100% identity between mouse and human amino acid sequence!) and a selection marker using calcium phosphate procedure. Control cells were infected with pLenti6/V5-DEST carrying no transgene. Briefly, exponentially growing packaging cells were seeded at a density of 2.800.000 cells per T75-flask in 8 ml DMEM+10% FCS two days before transfection. 10 min before transfection chloroquine was added directly to the overlying medium (25 μM end concentration). A 2 ml transfection mix consisting of 20 μg plasmid-DNA (candidate:helper-viruses in a 1:1 ratio) and 250 mM CaCl₂ was prepared in a 15 ml plastic tube. The same volume of 2×HBS (280 μM NaCl, 50 μM HEPES, 1.5 mM Na₂HPO₄, pH 7.06) was added and air bubbles were injected into the mixture for 15 sec. The transfection mix was added drop wise to the packaging cells, distributed and the cells were incubated at 37° C., 5% CO₂ for 6 hours. The cells were washed with PBS and the medium was exchanged with 8 ml DMEM+10% CS per T75-flask. The cells were incubated for 2 days of virus collection at 37° C., 5% CO₂. The supernatant was then filtered through a 0.45 μm cellulose acetate filter and polybrene (end concentration 8 μg/ml) was added. Mammalian fibroblast (SGBS) cells in a sub-confluent state were overlaid with the prepared virus containing medium. The infected cells were selected for at least 2 weeks with 5 μg/ml blasticidin. Following selection the cells were checked for transgene expression by western blot and immunofluorescence. Over expressing cells were seeded for differentiation.

SGBS cells were maintained as fibroblasts and differentiated into adipocytes as described in the prior art and supra (see also Hauner et al., 2001). For analysing the role of the proteins disclosed in this invention in vitro assays for the determination of free fatty acid, glucose uptake and triglyceride storage (see above) were performed.

Cellular Free Fatty Acid Uptake by Differentiated Adipocytes (SGBS Cells); (FIG. 9A)

During the terminal stage of adipogenesis (D12) cells were analysed for their ability to transport long chain fatty add across the plasma membrane. A modified protocol to the method of Abumrad et al. ((1991) Proc. Natl. Acad. Sci. USA 88: 6008-6012) for cellular transportation of fatty acid was established. In summary, cells were washed 3 times with PBS prior to serum starvation. This was followed by incubation in KRBH buffer, supplemented with 0.1% FCS for 2.5 h at 37° C. Uptake of exogenous free fatty acids was initiated by the addition of isotopic media containing non radioactive oleate and (³H) oleate (NEN Life Sciences) complexed to serum albumin in a final activity of 1 μCi/Well/ml in the presence of 5 mM glucose for 30 min at room temperature (RT). For the calculation of passive diffusion (PD) in the absence of active transport (AT) across the plasma membrane 20 mM of phloretin in glucose free media (Sigma) was added for 30 min at room temperature (RT). All assays were performed in duplicate wells. To terminate the active transport 20 mM of phloretin in glucose free media was added to the cells. Cells were lysed in 1 ml 0.1N NaOH and the protein concentration of each well were assessed using the standard Biuret method (Protein assay reagent; Bio-Rad).

We found that active transport of exogenous fatty acids across the plasma membrane of Prl-1 overexpressing cells were significantly higher at day 9 and other days (d7 and d12; data not shown) of adipogenesis when compared to control cells (FIG. 9A). This is in line with the described increase of triglyceride content in these cells (see above).

Cellular Glucose Uptake by Differentiated Adipocytes (SGBS Cells); (FIG. 9B)

For the determination of glucose uptake, cells were washed 3 times with PBS prior to serum starvation in Krebs-Ringer-Bicarbonat-Hepes buffer (KRBH; 134 mM NaCl, 3.5 mM KCl, 1.2 mM KH₂ PO₄, 0.5 mM MgSO₄, 1.5 mM CaCl₂, 5 mM NaHCO₃, 10 mM Hepes, pH 7.4), supplemented with 0.1% BSA and 0.5 mM Glucose for 2.5 h at 37° C. For insulin-stimulated glucose uptake, cells were incubated with 20 nM human insulin (Sigma; carrier: 10 mM HCl; 1% BSA) for 45 min at 37° C. Basal glucose uptake was determined with carrier only. Non-metabolizable 2-Deoxy-³H-D-Glucose (NEN Life Science, Boston, USA) in a final activity of 0.4 μCi/Well/ml was added for 30 min at 37° C. For the calculation of background radioactivity, 25 μM Cytochalasin B (Sigma) was used. All assays were performed in triplicate wells. To terminate the reaction, cells were washed 3 times with ice cold PBS, and lysed in 320 μl 0.1N NaOH. Protein concentration of each well was assessed using the Bio-Rad DC Protein assay reagent (Bio-Rad), and scintillation counting of cell lysates in 5 ml Ultima-gold cocktail (Packard Bioscience, Groningen, Netherlands) was performed.

As shown in FIG. 9B the insulin-stimulated glucose uptake of SGBS cells over-expressing Prl-1 is increased by about 100% during adipogenesis. This effect is already visible in the differentiating adipocyte (d9) but is also true for a fully differentiated adipocyte (d12, data not shown). This increase in glucose and therefore energy uptake of the cells is most likely the reason for the increased triglyceride levels we observed during SGBS differentiation (discussed above). Prl-1 does not seem to influence basal glucose uptake in a significant manner, but clearly has an effect on the glucose uptake of adipocytes.

Taken together the over-expression of Prl-1 showed an effect on metabolism of exogenous free fatty acids and glucose as well as triglyceride storage in all three assays we performed in SGBS cells, making it a potential interesting drug target for the treatment of diabetes and related metabolic disorders.

### Example 6

### Assays for the Determination of Lipid Storage, Synthesis and Transport of Prl-1 LOF Adipocytes (FIG. 6)

Loss of Function in 3T3-L1 Adipocytes by RNAi Technique

In order to stably inhibit Prl-1 expression, 3T3-L1 preadipocytes were engineered by retroviral infection aimed to express a target specific short interfering RNA construct under the control of the human hH1 promoter according to Brummelkamp et al. (Science 2002, Vol 296, p. 550-553). The following Prl-1 specific RNAi sequence was used: AGG ATT CCA ATG GTC ATA G (SEQ ID NO. 14).

Retroviral Infection of Preadipocytes

Packaging cells were transfected with a retroviral plasmid pLPCX carrying the specific RNAi construct under the control of the human hH1 promoter and a selection marker using calcium phosphate procedure. Control cells were infected with the same vector carrying no transgene.

Briefly, exponentially growing packaging cells were seeded at a density of 350,000 cells per 6-well in 2 ml DMEM+10% FCS one day before transfection. 10 min before transfection chloroquine was added directly to the overlying medium (25 μM final concentration). A 250 μl transfection mix consisting of 5 μg plasmid-DNA (candidate:helper-virus in a 1:1 ratio) and 250 mM CaCl₂ was prepared in a 15 ml plastic tube. The same volume of 2×HBS (280 μM NaCl, 50 μM HEPES, 1.5 mM Na₂HPO₄, pH 7.06) was added and air bubbles were injected into the mixture for 15 sec. The transfection mix was added drop wise to the packaging cells, distributed and the cells were incubated at 37° C., 5% CO₂ for 6 hours. The cells were washed with PBS and the medium was exchanged with 2 ml DMEM+10% CS per 6-well. One day after transfection the cells were washed again and incubated for 2 days of virus collection in 1 ml DMEM+10% CS per 6-well at 32° C., 5% CO₂. The supematant was then filtered through a 0.45 μm cellulose acetate filter and polybrene (final concentration 8 μg/ml) was added. Mammalian fibroblast (3T3-L1) cells in a sub-confluent state were overlaid with the prepared virus containing medium. The infected cells were selected for 1 week with 2 μg/ml puromycin.

3T3-L1 cells were maintained as fibroblasts and differentiated into adipocytes as described in the prior art and supra. The reduction of expression was more than 60% during differentiation as verified by quantitative rtPCR. For analysing the role of the proteins disclosed in this invention in the in vitro assays for the determination of lipid storage, synthesis and transport were performed.

Synthesis of Lipids During Adipogenesis (FIG. 6A)

During adipogenesis (day 06) cells were analysed for their ability to metabolise lipids. A modified protocol to the method of Jensen et al. ((2000) JBC 275: 40148) for lipid synthesis was established. Cells were washed 3 times with PBS prior to serum starvation in Krebs-Ringer-Bicarbonate-Hepes buffer (KRBH; 134 nM NaCl, 3.5 mM KCl, 1.2 mM KH₂ PO₄, 0.5 mM MgSO₄, 1.5 mM CaCl₂, 5 mM NaHCO₃, 10 mM Hepes, pH 7.4), supplemented with 0.1% FCS. for 2.5 h at 37° C. For insulin-stimulated lipid synthesis, cells were incubated with 1 μM bovine insulin (Sigma; carrier: 0.005 N HCl) for 45 min at 37° C. Basal lipid synthesis was determined with carrier only. ¹⁴C(U)-D-Glucose (NEN Life Sciences) in a final activity of 1 μCi/Well/ml in the presence of 5 mM glucose was added for 30 min at 37° C. For the calculation of background radioactivity, 25 μM Cytochalasin B (Sigma) was used. All assays were performed in triplicate wells. To terminate the reaction, cells were washed 3 times with ice cold PBS, and lysed in 1 ml 0.1 N NaOH. Protein concentration of each well was assessed using the standard Biuret method (Protein assay reagent; Bio-Rad). Total lipids were separated from aqueous phase after overnight extraction in Insta-Fluor scintillation cocktail (Packard Bioscience) followed by scintillation counting.

Our results show that Prl-1 LOF cells were less effective at synthesising lipids from exogenous glucose when stimulated with insulin compared to controls (FIG. 6A). The level of fatty acid esters in Prl-1 LOF cells was considerably higher at day 12 of adipogenesis when compared to control cells (FIG. 6B).

### Example 7

### Analysis of the Differential Expression of Transcripts of the Proteins of the Invention in Human Tissues

RNA preparation from human primary adipose tissues was done as described in Example 4. The target preparation, hybridization, and scanning was performed as described in the manufactures manual (see Affymetrix Technical Manual, 2002, obtained from Affymetrix, Santa Clara, USA).

In FIGS. 7A-B, the X-axis represents the time axis, shown are day 0 and day 12 of adipocyte differentiation. The Y-axis represents the flourescent intensity. The expression analysis (using Affymetrix GeneChips) of the protein tyrosine phosphatase type IVA, member 1 (PRL-1) gene using human abdominal derived primary adipocyte differentiation and human adipocyte cell line (SGBS) differentiation, clearly shows differential expression of the human PRL-1 gene in adipocytes. Several independent experiments were done. The experiments further show that the PRL-1 transcript (see FIGS. 7A-B) are most abundant at day 0 compared to day 12 during differentiation.

Thus, the PRL-1 protein has to be significantly decreased in order for the preadipocyctes to differentiate into mature adipocycte. Therefore, PRL-1 in preadipocyctes has the potential to inhibit adipose differentiation. Therefore, the PRL-1 protein might play an essential role in the regulation of human metabolism, in particular in the regulation of adipogenesis and thus it might play an essential role in obesity, diabetes, and/or metabolic syndrome.

### Example 8

### Generation and Analysis of Prl-1, Prl-2, or Prl-3 Transgenic Mice

Generation of the Transgenic Animals

Mouse Prl-1, Prl-2, and Prl-3 cDNA was isolated from mouse brown adipose tissue (BAT) using standard protocols as known to those skilled in the art. The cDNA was amplified by RT-PCR and point mutations were introduced into the cDNA.

The resulting mutated cDNA was cloned into a suitable transgenic expression vector. The transgene was microinjected into the male pronucleus of fertilized mouse embryos (preferably strain C57/BL6/CBA F1 (Harlan Winkelmann). Injected embryos were transferred into pseudo-pregnant foster mice. Transgenic founders were detected by PCR analysis. Two independent transgenic mouse lines containing the construct were established and kept on a C57/BL6 background. Briefly, founder animals were backcrossed with C57/BL6 mice to generate F1 mice for analysis. Transgenic mice were continuously bred onto the C57/Bl6 background. The expression of the protein of the invention can be analyzed by Taqman analysis as described above, and further analysis of the mice can be done as known to those skilled in the art.

For the purpose of the present invention, it will be understood by the person having average skill in the art that any combination of any feature mentioned throughout the specification is explicitly disclosed herewith.

1. A pharmaceutical composition comprising a PRL-1 homologous protein or/and a functional fragment thereof, a nucleic acid molecule encoding a PRL-1 homologous protein or/and a functional fragment thereof or/and a modulator/effector of said nucleic acid molecule or/and protein together with pharmaceutically acceptable carriers, diluents or/and additives.

2. The composition of claim 1, wherein the nucleic acid molecule is a vertebrate or insect PRL-1 nucleic acid, particularly encoding the human PRL-1 homologs (such as human Prl-1, Prl-2, or Prl-3 protein), and/or a nucleic molecule which is complementary thereto or a functional fragment thereof or a variant thereof.

3. The composition of claim 1, wherein said nucleic acid molecule is selected from the group of
(a) a nucleic acid molecule encoding a polypeptide as shown in Table 2 or/and an isoform, fragment, or/and variant of said polypeptide;
(b) a nucleic acid molecule which comprised or is the nucleic acid molecule as shown in Table 2;
(c) a nucleotide sequence which hybridizes at 50° C. in a solution containing 1×SSC and 0.1% SDS to a sequence of (a) or (b),
(d) a nucleic acid molecule being degenerated as a result of the genetic code to the nucleic acid sequence as defined in (a), (b) or (c);
(e) a nucleic acid molecule that encodes a polypeptide which is at least 85%, preferably at least 90%, more preferably at least 95%, more preferably at least 98% and up to 99.6% identical to the human PRL-1 homologous protein, preferably as described in Table 2 or as defined in claim 2 or to a polypeptide as defined in (a);
(f) a nucleic acid molecule that differs from the nucleic acid molecule of (a) to (e) by mutation and wherein said mutation causes an alteration, deletion, duplication or premature stop in the encoded polypeptide; and
(g) a partial sequence of any of the nucleotide sequences of (a) to (e) having a length of 15-25 bases, preferably 25-35 bases, more preferably 35-50 bases and most preferably at least 50 bases.

4. The composition of claim 1, wherein the nucleic acid molecule is a DNA molecule, particularly a cDNA or a genomic DNA.

5. The composition of claim 1, wherein said nucleic acid encodes a polypeptide contributing to regulating the energy homeostasis and/or the metabolism of triglycerides.

6. The composition of claim 1, wherein said nucleic acid molecule is a recombinant nucleic acid molecule.

7. The composition of claim 1, wherein the nucleic acid molecule is a vector, particularly an expression vector.

8. The composition of claim 1, wherein the polypeptide is a recombinant polypeptide.

9. The composition of claim 8, wherein said recombinant polypeptide is a fusion polypeptide.

10. The composition of claim 1, wherein said nucleic acid molecule is selected from hybridization probes, primers and anti-sense oligonucleotides.

11. The composition of claim 1 which is a diagnostic composition.

12. The composition of claim 1 which is a therapeutic composition.

13. The composition of claim 1 for the manufacture of an agent for detecting or/and verifying, for the treatment, alleviation and/or prevention of metabolic diseases or dysfunctions, including metabolic syndrome, obesity or/and diabetes, as well as related disorders such as eating disorder, cachexia, hypertension, coronary heart disease, hypercholesterolemia, dyslipidemia, osteoarthritis, gallstones, or liver fibrosis, in cells, cell masses, organs and/or subjects.

14. The composition of claim 1 for application in vivo.

15. The composition of claim 1 for application in vitro.

16. Use of a nucleic acid molecule encoding a PRL-1 homologous protein or an isoform, a functional fragment or variant thereof, in particular a nucleic acid molecule as described in Table 2, particularly of a nucleic acid molecule according to claim 3(a), (b), or (c), or/and a polypeptide encoded thereby or/and a functional fragment or/and a variant of said nucleic acid molecule or said polypeptide or/and a modulator/effector of said nucleic acid molecule or polypeptide for the manufacture of a medicament for the treatment of obesity, diabetes, or/and metabolic syndrome for controlling the function of a gene or/and a gene product which is influenced or/and modified by a PRL-1 homologous polypeptide.

17. Use of the nucleic acid molecule encoding a PRL-1 homologous protein or an isoform, a functional fragment or variant thereof, in particular a nucleic acid molecule as described in Table 2, particularly of a nucleic acid molecule according to claim 3(a), (b), or (c), or/and a polypeptide encoded thereby or/and a functional fragment or/and a variant of said nucleic acid molecule or said polypeptide or/and a modulator/effector of said nucleic acid molecule or said polypeptide for identifying substances capable of interacting with a PRL-1 homologous polypeptide, particularly with a polypeptide according to claim 3.

18. A non-human transgenic animal exhibiting a modified expression of a PRL-1 homologous polypeptide, particularly of a polypeptide according to claim 3.

19. The animal of claim 18, wherein the expression of the PRL-1 homologous polypeptide is increased and/or reduced.

20. A recombinant host cell exhibiting a modified expression of a PRL-1 homologous polypeptide, particularly of a polypeptide according to claim 3.

21. The cell of claim 20 which is a human cell.

22. A* method of identifying a (poly)peptide involved in the regulation of energy homeostasis and/or metabolism of triglycerides in a mammal comprising the steps of
(a) contacting a collection of (poly)peptides with a PRL-1 homologous polypeptide, particularly a polypeptide according to claim 3, or a functional fragment thereof under conditions that allow binding of said (poly)peptides;
(b) removing (poly)peptides which do not bind and
(c) identifying (poly)peptides that bind to said PRL-1 homologous polypeptide.

23. A method of screening for an agent which modulates/effects the interaction of a PRL-1 homologous polypeptide, particularly of a polypeptide according to claim 3, with a binding target/agent, comprising the steps of
(a) incubating a mixture comprising
(aa) a PRL-1 homologous polypeptide, particularly a polypeptide according to claim 3, or a functional fragment thereof;
(ab) a binding target/agent of said polypeptide or functional fragment thereof; and
(ac) a candidate agent under conditions whereby said polypeptide or functional fragment thereof specifically binds to said binding target/agent at a reference affinity;
(b) detecting the binding affinity of said polypeptide or functional fragment thereof to said binding target to determine an affinity for the agent; and
(c) determining a difference between affinity for the agent and the reference affinity.

24. A method of screening for an agent, which modulates/effects the activity of a PRL-1 homologous polypeptide, particularly of a polypeptide according to claim 3, comprising the steps of
(a) incubating a mixture comprising
(aa) said polypeptide or a functional fragment thereof, and
(ab) a candidate agent under conditions whereby said polypeptide or fragment thereof has a reference activity;
(b) detecting the activity of said polypeptide or functional fragment thereof to determine an activity in presence of the agent; and
(c) determining a difference between the activity in the presence of the agent and the reference activity.

25. A method of producing a composition comprising mixing the (poly)peptide identified by the method of claim 22 with a pharmaceutically acceptable carrier, diluent or/and additive.

26. The method of claim 25 wherein said composition is a pharmaceutical composition for preventing, alleviating or/and treating of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

27. Use of a (poly)peptide as identified by the method of claim 22 for the preparation of a pharmaceutical composition for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

28. Use of a nucleic acid molecule as defined in claim 1 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

29. Use of a polypeptide as defined in claim 1 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

30. Use of a vector as defined in claim 7 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

31. Use of a host cell as defined in claim 20 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

32. Use of a PRL-1 homologous nucleic acid molecule or/and of a functional fragment thereof for the production of a non-human transgenic animal which over- or under-expresses the PRL-1 homologous gene product.

33. Kit comprising at least one of
(a) a PRL-1 homolgous nucleic acid molecule or/and a functional fragment thereof;
(b) a PRL-1 homologous amino acid molecule or/and a functional fragment or/and an isoform thereof;
(c) a vector comprising the nucleic acid of (a);
(d) a host cell comprising the nucleic acid of (a) or the vector of (c);
(e) a polypeptide encoded by the nucleic acid of (a);
(f) a fusion polypeptide encoded by the nucleic acid of (a);
(g) an antibody, an aptamer or/and another modulator/effector of the nucleic acid of (a) or the polypeptide of (b), (e), or/and (f) and
(h) an anti-sense oligonucleotide of the nucleic acid of (a).

34. A method of producing a composition comprising mixing the agent identified by the method of claim 23 with a pharmaceutically acceptable carrier, diluent or/and additive.

35. A method of producing a composition comprising mixing the agent identified by the method of claim 24 with a pharmaceutically acceptable carrier, diluent or/and additive.

36. Use of an agent as identified by the method of claim 23 for the preparation of a pharmaceutical composition for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

37. Use of an agent as identified by the method of claim 24 for the preparation of a pharmaceutical composition for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.


SEQUENCE LISTING (26 sequences)

SEQ ID NO: 1 - DNA, 4394 bp, Homo sapiens
ccggctcggtacgcgctctgctccgagccgctcactgcgtggtagagtctggtgcccccgccgtcgcctgcatcgccgccaccgccgctccgccacgaccaccgccgcctcctgccctgcagccaccgccaccgcctgtgtcgccgccgcctcgggaccggctgtatgattaggccacaatcttcaatgagtaaacatattcctcaattctgtggtgttcttggtcacacatttatggagtttctgaagggcagtggagattactgccaggcacagcacgacctctatgcagacaagtgaactgtagaaactgattactgctccaccaagaagcccccataagagtggttatcctggacacagaagtgttgaattgaaatccacagagcattttacaagagttctgacctggatggggtaaacctcagtgcacttcttttctgttggcctcagtattactggattgaagaattgctgcttcttgttaggaggttcatttcacttatcattacttacaacttcatactcaaagcactgagaatttcaagtggagtatattgaagtagacttcagtttctttgcatcatttctgtattcaatttttttaattatttcataaccctattgagtgttttttaactaaattaacatggctcgaatgaaccgcccagctcctgtggaagtcacatacaagaacatgagatttcttattacacacaatccaaccaatgcgaccttaaacaaatttatagaggaacttaagaagtatggagttaccacaatagtaagagtatgtgaagcaacttatgacactactcttgtggagaaagaaggtatccatgttcttgattggccttttgatgatggtgcaccaccatccaaccagattgttgatgactggttaagtcttgtgaaaattaagtttcgtgaagaacctggttgttgtattgctgttcattgcgttgcaggccttgggagagctccagtacttgttgccctagcattaattgaaggtggaatgaaatacgaagatgcagtacaattcataagacaaaagcggcgtggagcttttaacagcaagcaacttctgtatttggagaagtatcgtcctaaaatgcggctgcgtttcaaagattccaacggtcatagaaacaactgttgcattcaataaaattggggtgcctaatgctactggaagtggaacttgagatagggcctaatttgttatacatattagccaacatgttggcttagtaagtctaatgaagcttccataggagtattgaaaggcagttttaccaggcctcaagctagacagatttggcaacctctgtatttgggttacagtcaacctatttggatacttggcaaaagattcttgctgtcagcatataaaatgtgcttgtcatttgtatcaattgacctttccccaaatcatgcagtattgagttatgacttgttaaatctattcccatgccagaatcttatcaatacataagaaatttaggaagattaggtgccaaaatacccagcacaatacttgtatatttttagtaccatacagaagtaaaatcccaggaactatgaacactagaccttatgtggtttattccttcaatcatttcaaacattgaaagtagggcctacatggttatttgcctgctcactttatgtttacatctcccacattcataccaatatacgtcaggtttgcttaaccattgatttttttttttttttaccaagtcttacagtgattattttacgtgtttccatgtatctcactttgtgctgtattaaaaaaacctccattttgaaaatctacgttgtacagaagcacatgtctttaatgtcttcagacaaaaaagccttacattaatttaatgtttgcactctgaggtgcaacttaacagggagggcctgagaaaagaatgggagggggctattaattattttttagcaaaatgttgcctttgtcttgtgcaaacatgtagaatatgctctttaatttagtaaaatatttttttaaaaggtagagatgctttgttattgtaatcataaacttcctgaaattcttgtaatttttttcccatacttatcagaagtgtgtttaccaacttatttttgtttgaaagtgtgattttttttttccttcccaacctctcttgcaaaaaaagaaatgggtttctgctaatgaattgagcacatctaatattttatatgccttttggagctgtgtaagttaatatttgatacttgacaatttgttttattatgtaattgataaaatggtgatgtgtattaatgttagttcaaccatatatttatactgtctggggatgtgtggttatagttctgtgggagaaataattttgtcagtgttcaccagcttgtaaaaacttagtgcgagagctgaaacatctaaataaataatgacatgcatttatcatcattgagattggtttgctttaaattaacttattttgtagaagacaaaatgaattgcacttcacttaatgtgtgtcctcatctttttacaaataaatgaaggattataaatgatgtcagcattttagtaaacttttagacaaaatttgttagggtcattcatgaaaactttaatactaaaagcactttccattatatactttttaaaggtctagataattttgaaccaatttattattgtgtactgaggagaaataatgtatagtagaggacagccttggtttgtaaagctcagttccactagttcatggttttgtgcaacttctgagcctcagttttctcctttgcaaattaataattacatacctttatagattttgaaattaatttaaatattagtatttggtacatgaaggcttaatgttaagtttcctttaatgatccacaataatccctttgatcacgttaatctaaatctagatgtctttgtctaattttttttgaatagcagttataaatgtaaaggactcaaagtttaagtaaaaagtgatactccaccttgtgtttcaaagaatttagttccacctcttcataccagtttaacacttaatatatttcattggattttagacagggcaaaaggaagaacaggggcctctggaggcccttggttatttaaatcttggattatttgtgatagtaatcacaaatttttggctaatttttaacctgaggttttgttttttttttaaaggaaatgcagcctagtcttgagaacataattttatataatcaattactaaatgttaaactattaccacacagcccataaaacagcatttgcgtttattgagagagaggatgtgccatcatgattaatgaaaactatcttttgagtttgaaaagaaattaatttgcagtgtttggattgtatatatggtgctaaaaataaattaatttactttataaaccttatctgtacattatacgatgtgatgaaatttgctttttatccaaatattttgtatcttgtaaatatggctaattataggaatgcctataatacatcttagattccttatatctaataagagttcaaagagttatgagttgaagtcttgaatgcaggaaactatctgatagtgttctaaaatttggttacttgggtttggatacccttagtgggatgatgtaaatagaggctagctacctaggcttgtctatagcaaccataatgttgatgtaagtaatgcggttactgaatcataagaaaatgccatctctttttagttgaaggaaaactctggaagtaggtgccattggtcattctgcagtgcactgcaaccattgtttcccctagtgccctcttttccctagggcattgctctcctattcccacgccttaacacagctctatacctagaagcagccagcccaggcatgcagtcacatttaatcacatcccccttctagagtgcttcaaaatgatgtagtccctcaacttggctaaagaatctcaatctcttgaaatttatttttttaatgtcatattcatctggtaaatatctactgtttgccaggcatttaagaatatggcaaagaacataaaagatggtgtcaccagattttggtcaccaatgagtacccgacccgttgccatgattaagagagaatgctttctattggagtttcaggaaatataatttgagaatactttaaagggaagtggaagtataagtgaatgatatttttcttttacatgtaaacaatgaagttatttcaaagttaagttttaaacaaaatacatgaagtagtgtctgccatacatgttaatattctacattcttgcttccttaaattaatatgtttgtgtgtatatatgtgcctcacacctgaattgaaaattaaagactggtttaaaagtggttaaaaaaaaaaaaaaaaaa

SEQ ID NO: 2 - protein, 173 aa, Homo sapiens
MARMNRPAPVEVTYKNMRFLITHNPTNATLNKFIEELKKYGVTTIVRVCEATYDTTLVEKEGIHVLDWPFDDGAPPSNQIVDDWLSLVKIKFREEPGCCIAVHCVAGLGRAPVLVALALIEGGMKYEDAVQFIRQKRRGAFNSKQLLYLEKYRPKMRLRFKDSNGHRNNCCIQ

SEQ ID NO: 3 - DNA, 3925 bp, Homo sapiens
agcggggctgcgcgaagtcatcgctgttccagacagcgatgactcgagagcggtgggggtggcggcgcgatcggccgggctgtaaccgtcgtctgtccgggagcggctggagcggcagcggcggccgggcacggcgcgaggtgacgccacagggcagcggcggcagcggaggcagcggcggcagcaggagacgcagcggcggccgcagcagcagcagcaagacggactcgtggagacgcgccgccgccgccgccgccgggccgggccgggtgtcgcgcgccgaggctgggggggagtcgtcgccgccgccgccaccgctaccgccgccgccgccgccgccgaggtgactgaggagagaggcgcctcctcgctcccgccaccgccggacttcaatgcccagtccccagctcgccagcgtttttcgttggaatatacgttgcacatttatggcgattctgagtgtgagggcagacttctgccaggctcagcacagcattttcgctgacaagtgagcttggaggttctatgtgccataattaacattgccttgaagactcctggacaccgagactggcctcagaaatagttggcttttttttttttttaattgcaagcatatttcttttaatgactccagtaaaattaagcatcaagtaaacaagtggaaagtgacctacacttttaacttgtctcactagtgcctaaatgtagtaaaggctgcttaagttttgtatgtagttggattttttggagtccgaatatttccatctgcagaaattgaggcccaaattgaatttggattcaagtggattctaaatactttgcttatcttgaagagagaagcttcataaggaataaacaagttgaatagagaaaacactgattgataataggcattttagtggtctttttaatgttttctgctgtgaaacatttcaagatttattgatttttttttttcactttccccatcacactcacacgcacgctcacactttttatttgccataatgaaccgtccagcccctgtggagatctcctatgagaacatgcgttttctgataactcacaaccctaccaatgctactctcaacaagttcacagaggaacttaagaagtatggagtgacgactttggttcgagtttgtgatgctacatatgataaagctccagttgaaaaagaaggaatccacgttctagattggccatttgatgatggagctccaccccctaatcagatagtagatgattggttaaacctgttaaaaaccaaatttcgtgaagagccaggttgctgtgttgcagtgcattgtgttgcaggattgggaagggcacctgtgctggttgcacttgctttgattgaatgtggaatgaagtacgaagatgcagttcagtttataagacaaaaaagaaggggagcgttcaattccaaacagctgctttatttggagaaataccgacctaagatgcgattacgcttcagagataccaatgggcattgctgtgttcagtagaaggaaatgtaaacgaaggctgacttgattgtgccatttagagggaactcttggtacctggaaatgtgaatctggaatattacctgtgtcatcaaagtagtgatggattcagtactcctcaaccactctcctaatgattggaacaaaagcaaacaaaaaagaaatctctctataaaatgaataaaatgtttaagaaaagagaaagagaaaaggaattaattcagtgaaggatgattttgctcctagttttggagtttgaatttctgccaggattgaattattttgaaatctcctgtctttttaaactttttcaaaataggtctctaaggaaaaccagcagaacattaggcctgtgcaaaaccatctgtttggggagcacactcttccattatgcttggcacatagatctccctgtggtgggattttttttttccctttttttgtgggggagggttggtggtatatttttcccctcttttttccttcctctcctacatctcccttttcccccgatccaagttgtagatggaatagaagcccttgttgctgtagatgtgcgtgcagtctggcagccttaagcccacctgggcacttttagataaaaaaaaaaaaaaacaaaaaacaacaccaaaaaaacagcagtgatatatatattccaggtggtttttagtctttactgatgaaagggtgttcatgttagtttcttcaaaaccctatctaatactaggcaaagtagccaagagccttttgttttgtttttattttgataaattagtggagaaatggcattttaagaggagtctcttctcaacttacctgagagtcgaattcttctcttccctaaccaatgaagctaagtggttatcccagaaacttgtcttctaaaagggaggactccaggccatcaataaagatgtccaggcagtgagcgtactttttacaccctgtagaattgtgggctgtagcgttactctgattttctgtctagtatcagagaatgctggtagcttaaaatttttattttaggacttgtactctgaattttcaggaaccgtcaaaggagcagcagcaaattcacatattttcgacttgagaaatgcttgtggtatgtgttttccaaactgccccctatatgtaaagttcagtttaaccactgattgccttgttattactaggttttttgagattaaaaaaaaaaaatccctggtttaaaaccaacaatgatgcctagtgagtatgtgtccacaggccataacagggtagaagagagacatcgtgcaacccaatgagtagtgaagggactgtgttgcttgtgaagcggtgtagtagcatttttgcagattcttggctgggtttagtgtactgatctagaaaagctgtttttctgctcctttgtggaaggcagttatgatcaggctgcatggacaaagcaggtagaggggcaccatcaggggctcttgcactattttcacctctaaatattacgtactcagtagtgccctgcttctagggctctgaatacgggcttaaagtcatcttgtcctgctggaatttgctgtgcagagccataagcctcccattttgttagcgtcagctaggccaataggaacagaccgggaccttgtctcacactgatgatacctcacatgttgaccggctatgtgaactgcctatttcctatgctggagttttgatttttaactaaacgcaaatctgtagattctctcctctcccatcccagaaaacaaaacaaaataatgcttttcgaaattgtttctaggactttaaaacataatggtatatccaaaattctttatttcagaatgcaacaatagattccattaatatagactcaagatcaaaacagcatacctgctaagctaagatagatggtgttgattccactgggttttgatcaatacaataacaaacctttttcctttgacatactctgaattttgttgtttggggggagggggtgtgtgtgtgtgtgtgtgtgtgtgtgtgtattgtgtgtgtgtgtgtgtgcacgcgcagtgtccatcagtatcagtgcctgcctgagttaggaaaattacattcctggttctgtattgaggagaaggatgtataaagcaacatgaaacattagccctccttttattttaaagactaatgttaattgttcttaaaactggattttttttccttaaagcaatttttttcttttcgatttaatgaagtattgctagctgaagccagtttgacatagagagatgtcagattgatttgaaaggtgtgcagcctgatttaaaaccaaaccctgaacccttttaaagaacaataaaacatattttacacgctcaaaaaaaaaaaaaaaaaaaaaaaaaaaaaaaaaaaaaaaaaaaaaaaaaaaaaaaaaaaaaaaaaaa

SEQ ID NO: 4 - protein, 167 aa, Homo sapiens
MNRPAPVEISYENMRFLITHNPTNATLNKFTEELKKYGVTTLVRVCDATYDKAPVEKEGIHVLDWPFDDGAPPPNQIVDDWLNLLKTKFREEPGCCVAVHCVAGLGRAPVLVALALIECGMKYEDAVQFIRQKRRGAFNSKQLLYLEKYRPKMRLRFRDTNGHCCVQ

SEQ ID NO: 5 - DNA, 2084 bp, Homo sapiens
agcggggctgcgcgaagtcatcgctgttccagacagcgatgactcgagagcggtgggggtggcggcgcgatcggccgggctgtaaccgtcgtctgtccgggagcggctggagcggcagcggcggccgggcacggcgcgaggtgacgccacagggcagcggcggcagcggaggcagcggcggcagcaggagacgcagcggcggccgcagcagcagcagcaagacggactcgtggagacgcgccgccgccgccgccgccgggccgggccgggtgtcgcgcgccgaggctgggggggagtcgtcgccgccgccgccaccgctaccgccgccgccgccgccgccgaggtgactgaggagagaggcgcctcctcgctcccgccaccgccggacttcaatgcccagtccccagctcgccagcgtttttcgttggaatatacgttgcacatttatggcgattctgagtgtgagggcagacttctgccaggctcagcacagcattttcgctgacaagtgagcttggaggttctatgtgccataattaacattgccttgaagactcctggacaccgagactggcctcagaaatagttggcttttttttttttttaattgcaagcatatttcttttaatgactccagtaaaattaagcatcaagtaaacaagtggaaagtgacctacacttttaacttgtctcactagtgcctaaatgtagtaaaggctgcttaagttttgtatgtagttggattttttggagtccgaatatttccatctgcagaaattgaggcccaaattgaatttggattcaagtggattctaaatactttgcttatcttgaagagagaagcttcataaggaataaacaagttgaatagagaaaacactgattgataataggcattttagtggtctttttaatgttttctgctgtgaaacatttcaagatttattgatttttttttttcactttccccatcacactcacacgcacgctcacactttttatttgccataatgaaccgtccagcccctgtggagatctcctatgagaacatgcgttttctgataactcacaaccctaccaatgctactctcaacaagttcacagaggaacttaagaagtatggagtgacgactttggttcgagtttgtgatgctacatatgataaagctccagttgaaaaagaaggaatccacgttctagattggccatttgatgatggagctccaccccctaatcagatagtagatgattggttaaacctgttaaaaaccaaatttcgtgaagagccaggttgctgtgttgcagtgcattgtgttgcaggattgggaagggcacctgtgctggttgcacttgctttgattgaatgtggaatgaagtacgaagatgcagttcagtttataagacaaaaaagaaggggagcgttcaattccaaacagctgctttatttggagaaataccgacctaagatgcgattacgcttcagagataccaatgggcattgctgtgttcagtagaaggaaatgtaaacgaaggctgacttgattgtgccatttagagggaactcttggtacctggaaatgtgaatctggaatattacctgtgtcatcaaagtagtgatggattcagtactcctcaaccactctcctaatgattggaacaaaagcaaacaaaaaagaaatctctctataaaatgaataaaatgtttaagaaaagagaaagagaaaaggaattaattcagtgaaggatgattttgctcctagttttggagtttgaatttctgccaggattgaattattttgaaatctcctgtctttttaaactttttcaaaataggtctctaaggaaaaccagcagaacattaggcctgtgcaaaaccatctgtttggggagcacactcttggcacatagatctccctgtggtgggatttttttccctttttttgtgggggagggttggtggtatatttttcccctcttttttccttcctctcctacatctcccttttcccccgatccaagttgtagatggaatagaagcccttgttgctgtagatgtgcgtgcagtctggcagccttaagcccac

SEQ ID NO: 6 - protein, 167 aa, Homo sapiens; identical to SEQ ID NO: 4

SEQ ID NO: 7 - DNA, 1693 bp, Homo sapiens
agcggggctgcgcgaagtcatcgctgttccagacagcgatgactcgagagcggtgggggtggcggcgcgatcggccgggctgtaaccgtcgtctgtccgggagcggctggagcggcagcggcggccgggcacggcgcgaggtgacgccacagggcagcggcggcagcggaggcagcggcggcagcaggagacgcagcggcggccgcagcagcagcagcaagacggactcgtggagacgcgccgccgccgccgccgccgggccgggccgggtgtcgcgcgccgaggctgggggggagtcgtcgccgccgccgccaccgctaccgccgccgccgccgccgccgaggtgactgaggagagaggcgcctcctcgctcccgccaccgccggacttcaatgcccagtccccagctcgccagcgtttttcgttggaatatacgttgcacatttatggcgattctgagtgtgagggcagacttctgccaggctcagcacagcattttcgctgacaagtgagcttggaggttctatgtgccataattaacattgccttgaagactcctggacaccgagactggcctcagaaatagttggcttttttttttttttaattgcaagcatatttcttttaatgactccagtaaaattaagcatcaagtaaacaagtggaaagtgacctacacttttaacttgtctcactagtgcctaaatgtagtaaaggctgcttaagttttgtatgtagttggattttttggagtccgaatatttccatctgcagaaattgaggcccaaattgaatttggattcaagtggattctaaatactttgcttatcttgaagagagaagcttcataaggaataaacaagttgaatagagaaaacactgattgataataggcattttagtggtctttttaatgttttctgctgtgaaacatttcaagatttattgatttttttttttcactttccccatcacactcacacgcacgctcacactttttatttgccataatgaaccgtccagcccctgtggagatctcctatgagaacatgcgttttctgataactcacaaccctaccaatgctactctcaacaagttcacagaggaacttaagaagtatggagtgacgactttggttcgagtttgtgatgctacatatgataaagctccagttgaaaaagaaggaatccacgttctaaaaaagaaggggagcgttcaattccaaacagctgctttatttggagaaataccgacctaagatgcgattacgcttcagagataccaatgggcattgctgtgttcagtagaaggaaatgtaaacgaaggctgacttgattgtgccatttagagggaactcttggtacctggaaatgtgaatctggaatattacctgtgtcatcaaagtagtgatggattcagtactcctcaaccactctcctaatgattggaacaaaagcaaacaaaaaagaaatctctctataaaatgaataaaatgtttaagaaaagagaaagagaaaaggaattaattcagtgaaggatgattttgctcctagttttggagtttgaatttctgccaggattgaattattttgaaatctcctgtctttttaaactttttcaaaataggtctctaaggaaaaccagcagaacattaggcctgtgcaaaaccatctgtttggggagcacactctt

SEQ ID NO: 8 - protein, 82 aa, Homo sapiens
MNRPAPVEISYENMRFLITHNPTNATLNKFTEELKKYGVTTLVRVCDATYDKAPVEKEGIHVLKKKGSVQFQTAALFGEIPT

SEQ ID NO: 9 - DNA, 1396 bp, Homo sapiens
tgactatccagctctgagagacgggagtttggagttgcccgctttactttggttgggttggggggggcggcgggctgttttgttccttttcttttttaagagttgggttttcttttttaattatccaaacagtgggcagcttcctcccccacacccaagtatttgcacaatatttgtgcggggtatgggggtgggtttttaaatctcgtttctcttggacaagcacagggatctcgttctcctcattttttgggggtgtgtggggacttctcaggtcgtgtccccagccttctctgcagtcccttctgccctgccgggcccgtcgggaggcgccatggctcggatgaaccgcccggccccggtggaggtgagctacaaacacatgcgcttcctcatcacccacaaccccaccaacgccacgctcagcaccttcattgaggacctgaagaagtacggggctaccactgtggtgcgtgtgtgtgaagtgacctatgacaaaacgccgctggagaaggatggcatcaccgttgtggactggccgtttgacgatggggcgcccccgcccggcaaggtagtggaagactggctgagcctggtgaaggccaagttctgtgaggcccccggcagctgcgtggctgtgcactgcgtggcgggcctgggccgggctccagtccttgtggcgctggcgcttattgagagcgggatgaagtacgaggacgccatccagttcatccgccagaagcgccgcggagccatcaacagcaagcagctcacctacctggagaaataccggcccaaacagaggctgcggttcaaagacccacacacgcacaagacccggtgctgcgttatgtagctcaggaccttggctgggcctggtcgtcatgtaggtcaggaccttggctggacctggaggccctgcccagccctgctctgcccagcccagcaggggctccaggccttggctggccccacatcgccttttcctccccgacacctccgtgcacttgtgtccgaggagcgaggagcccctcgggccctgggtggcctctgggccctttctcctgtctccgccactccctctggcggcgctggccgtggctctgtctctctgaggtgggtcgggcgccctctgcccgccccctcccacaccagccaggctggtctcctctagcctgtttgttgtggggtgggggtatattttgtaaccactgggcccccagcccctcttttgcgaccccttgtcctgacctgttctcggcaccttaaattattagaccccggggcagtcaggtgctccggacacccgaaggcaataaaacaggagccgtgaaaaaaaaaaaaaaaaaaaaaaaaaaaaaaaaaaaaaaaaaaaaaaaaaaaaaaaaaaaaaaaa

SEQ ID NO: 10 - protein, 173 aa, Homo sapiens
MARMNRPAPVEVSYKHMRFLITHNPTNATLSTFIEDLKKYGATTVVRVCEVTYDKTPLEKDGITVVDWPFDDGAPPPGKVVEDWLSLVKAKFCEAPGSCVAVHCVAGLGRAPVLVALALIESGMKYEDAIQFIRQKRRGAINSKQLTYLEKYRPKQRLRFKDPHTHKTRCCVM

SEQ ID NO: 11 - DNA, 23 bp, Mus musculus
gctgtattgctgtccattgtgtc

SEQ ID NO: 12 - DNA, 23 bp, Mus musculus
tccaccttcaattaatgctaggg

SEQ ID NO: 13 - DNA, 21 bp, Mus musculus
  misc_feature (1)..(1): labeled with FAM reporter dye
caggccttggcagagctccgg

SEQ ID NO: 14 - DNA, 17 bp, Mus musculus
agctaccggcacatgcg

SEQ ID NO: 15 - DNA, 17 bp, Mus musculus
acgtgctgagggtggca

SEQ ID NO: 16 - DNA, 23 bp, Mus musculus
  misc_feature (1)..(1): labeled with FAM reporter dye
tcctcatcacccacaaccccagc

SEQ ID NO: 17 - DNA, 23 bp, Homo sapiens
tcgtgaagaacctggttgttgta

SEQ ID NO: 18 - DNA, 25 bp, Homo sapiens
ttaatgctagggcaacaagtactgg

SEQ ID NO: 19 - DNA, 23 bp, Homo sapiens
  misc_feature (1)..(1): labeled with FAM reporter dye
tgctgttcattgcgttgcaggcc

SEQ ID NO: 20 - DNA, 21 bp, Homo sapiens
ggagtgacgactttggttcga

SEQ ID NO: 21 - DNA, 23 bp, Homo sapiens
gccaatctagaacgtggattcct

SEQ ID NO: 22 - DNA, 38 bp, Homo sapiens
  misc_feature (1)..(1): labeled with FAM reporter dye
ttgtgatgctacatatgataaagctccagttgaaaaag

SEQ ID NO: 23 - DNA, 20 bp, Homo sapiens
aagtacgaggacgccatcca

SEQ ID NO: 24 - DNA, 20 bp, Homo sapiens
ctgcttgctgttgatggctc

SEQ ID NO: 25 - DNA, 20 bp, Homo sapiens
  misc_feature (1)..(1): labeled with FAM reporter dye
ttcatccgccagaagcgccg

SEQ ID NO: 26 - DNA, 19 bp, Artificial sequence
  RNAi construct with homology to homo sapiens Prl-1
aggattccaatggtcatag